(19) United States
(12) Patent Application Publication
(10) Pub. No.: US 2016/0370385 A1
(43) Pub. Date: Dec. 22, 2016

(54) ASSAY FOR ANTI-MULLERIAN HORMONE

(71) Applicant: OTAGO INNOVATION LIMITED, Dunedin (NZ)

(72) Inventors: Ian Stuart McLennan, Dunedin (NZ); Michael William Pankhurst, Dunedin (NZ)

(21) Appl. No.: 14/900,581

(22) PCT Filed: Jun. 20, 2014
(86) PCT No.: PCT/NZ2014/000123

§ 371 Date: Dec. 21, 2015

Related U.S. Application Data
(60) Provisional application No. 61/838,053, filed on Jun. 21, 2013.
(60) Provisional application No. 61/871,025, filed on Aug. 28, 2013.

(51) Int. Cl. G01N 33/74

(52) U.S. Cl. CPC G01N 33/74

(57) ABSTRACT

The present invention provides assays, methods and kits for detection and measurement of the different species of AMH present in a sample, and in particular a biological sample such as a whole blood sample, plasma or serum. The species of AMH include, for example, proAMH and AMH_{N,C}.

### TECHNICAL FIELD

The present invention provides assays, methods and kits for measuring, for example, an amount of an Anti-Müllerian Hormone (AMH) in a sample. In particular, the present invention provides assays, methods and kits for measuring different forms of AMH, including, for example, proAMH and AMH_{N,C}, in biological samples obtained from human and non-human animals.

### BACKGROUND OF THE INVENTION

The following includes information that may be useful in understanding the present invention. It is not an admission that any of the information, publications or documents specifically or implicitly referred to or referenced herein is prior art, or essential, to the presently described or claimed inventions. All publications and patents mentioned in this specification are incorporated herein by reference in their entirety.

The hormonal concentration of Anti-Müllerian Hormone (AMH; also known as Müllerian inhibiting Substance or MIS) varies with sex and age. AMH is initially male specific, with male embryos and boys having high levels of AMH in their blood (Lee et al. (1996) J Clin Endocrinol Metab 81:571-576; Aksglaede et al. (2010) J Clin Endocrinol Metab 95:5357-5364). AMH triggers the degeneration of the uterine precursor in early male embryos, but its presence throughout male development points to it having mulltple functions, some of which may underlie the male biases in developmental disorders. Consistent with this, AMH contributes to the virilisation of the brain and behaviour of mice, via a hormonal mechanism (Morgan et al. (2011) Behav Brain Res 221:304-306; Wang et al. (2009) Proc Natl Acad Sci USA 106:7203-7208; Wittmann & McLennan (2011) J Neuroendocrinol 23:831-838). In humans, a boy's level of AMH inversely correlates with a measure of his maturation (Morgan et al. (2011) PLoS One 6:e20533) and with the severity of his symptoms, if he has an autism spectrum disorder (Pankhurst & McLennan (2012) Transl Psychiatry 2:e148).

In young adults, men and women have similar levels of (total) AMH (Aksglaede et al. (2010) J Clin Endocrinol Metab 95:5357-5364; Laven et al. (2004) J Clin Endocrinol Metab 89:318-323). In women, the level of (total) AMH in blood decreases as the number of follicles in her ovaries diminishes (de Vet et al. (2002) Fertil Steril 77:357-362). Consequently, over 400 recent papers have investigated the use of blood AMH levels as a surrogate measure of ovarian reserve and/or ovarian pathology. AMH is the first ovarian secretion to cease during the perimenopausal transition (van Rooij et al. (2005) Fertil Steril 83:979-987). Hence, if AMH is a hormone in adults, then there are obvious implications for the understanding of menopause and the existence of sex biases in age-related conditions. The blood levels of AMH in adults are less than 10% of those in boys (Lee et al. (1996) J Clin Endocrinol Metab 81:571-576; Aksglaede et al. (2010) J Clin Endocrinol Metab 95:5357-5364); Laven et al. (2004) J Clin Endocrinol Metab 89:318-323). At first sight, this suggests that AMH in adult plasma lacks function. However, the adult levels of AMH are sufficient to modulate neuronal survival in vitro (Wang et al. (2005) Proc Natl Acad Sci USA 102:16421-16425) and to strongly activate AMH-reporters in some cell lines (Teixeira et al. (1999) Endocrinology 140:4732-4738; Allard et al. (2000) Development 127:3349-3360; Kim et al. (1992) J Clin Endocrinol Metab 75:911-917). The maintenance of blood vessels and Insulin homeostasis are emerging possibilities for the hormonal functions of AMH in adults (Robeva et al. (2012) Andrologla 44 Suppl 1:329-334; Dennis (2012) In: Anatomy. Dunedin: Otago University http://hdl.handle.net/10523/2558).

The abovementioned studies of AMH in adults and boys tacitly assume that there is a single molecular form of AMH in blood, which does not vary with age or gonadal pathology. A priori, this is not the case, as demonstrated by the experimental evidence and examples which follow.

AMH is synthesised as a glycosylated (Budzlk et al. (1980) Cell 21:909-915; Lorenzo et al. (2002) J Chromatogr B Biomed Sci Appl 766:89-98; Picard et al. (1986) FEBS Letters 195:73-76; Picard et al. (1978) Mol Cell Endocrinol 12:17-30) cysteine-linked homodimer of the 560 amino add preproprotein (preAMH) (Cate et al. (1986) Cell 45:685-698). The first 24 amino acids are removed during synthesis yielding the prohormone (proAMH) (Cate et al. (1986) Cell 45:685-698), which does not activate AMH receptors (Kurian et al. (1995) Clin Cancer Res 1:343-349; MacLaughlin et al. (1992) Endocrinology 131:291-296). Enzymatic cleavage at a monobasic recognition site between amino acids 451 and 452 (where amino add 1 is the start-coding methlonine) yields a 25 kDa C-terminal dimer (AMH_{C}) and 120 kDa N-terminal dimer (AMH_{N}), which initially remain associated in a non-colavent complex (AMH_{N,C}) (Pepinsky et al. (1988) J Biol Chem 263:18961-18964). AMH_{C} is the receptor-activating component of total AMH (MacLaughlin et al. (1992) Endocrinology 131:291-296; di Clemente et al. (2010) Mol Endocrinol 24:2193-2206). Some recombinant forms of AMH also contain alternatively cleaved AMH, which yields an extended C-terminal peptide consisting of amino acids 255-560 (AMH₂₅₅₋₅₆₀) (Ragin et al. (1992) Protein Expr Purf 3:236-245).

The AMH in blood is generally presumed to be the bioactive cleaved form based on an historic investigation of a sex-cord tumour patient with pathologically elevated levels of AMH (Ragin et al. (1992) Protein Expr Purif 3:236-245).

There are numerous publications which describe the detection and measurement of AMH, and in particular biologically active forms of AMH. For example, U.S. Pat. No. 7,897,350 describes compositions and methods to measure AMH in a sample, and antibodies that bind to the “mature” region or C-terminal domain of AMH. A purported benefit in the assay described in this patent is in providing stable and accurate measurement of AMH in samples, and particularly measurements which are unaffected by proteolysis or cleavage between the propeptide and mature hormone forms of AMH. European Patent Publication No. 2161579 describes a method for detecting or quantifying at least one biologically active form of AMH in a sample, such as cleaved AMH or C-terminal AMH, as well as antibodies which bind the same.

A number of other publications describe assays involving antibodies to the N-terminal domain of AMH. For example, Hudson et al. (1990) J. Clin. Endocrin. Metab. 70: 16-22 describes an immunoassay in which two monoclonal antibodies are directed against the pro or N-terminal domain of AMH. Long et al. (2000) J. Clin. Endocrin. Metab. 85:540-544 describe an Immunoassay which also uses two monoclonal antibodies; one which binds to an epitope in the N-terminal domain of AMH and the other which binds to an epitope in the C-terminal domain of AMH.

Despite the various advances in assays and methods for the detection and measurement of this hormone, the molecular form of AMH in blood has not been positively verified. Surprisingly, Applicants now provide the first direct evidence to show that the blood of boys, men and premenopausal women contain significant levels of both proAMH and AMH_{N,C}. AMH_{N,C} activates AMH receptors, whereas proAMH does not (Di Clemente et al. (2010) Mol Endocrinol 24:2193-2206). Assays that detect both species therefore report values that are an aggregate of two biologically distinct species, which may have different relationships to biological phenomena, such as ovarian reserve.

The present invention provides assays, methods and kits capable of detecting and measuring different forms of AMH in a biological sample, and in particular, proAMH and proAMH_{N,C}.

### BRIEF SUMMARY OF THE INVENTION

The Inventions described and claimed herein have many attributes and embodiments including, but not limited to, those set forth or described or referenced in this Brief Summary of the Invention. It is not intended to be all-inclusive and the inventions described and claimed herein are not limited to or by the features or embodiments identified in this Brief Summary of the Invention, which is included for purposes of Illustration only and not restriction.

Applicants provide the first direct evidence to show that different forms of Anti-Müllerian Hormone (AMH) exist in circulation, in particular proAMH and the biologically active form of the hormone AMH_{N,C}. Accordingly, the present invention provides assays, methods and kits for the detection and measurement of the different species of AMH present in a biological sample, and in particular a biological sample such as a whole blood sample, plasma and serum.

In one aspect of the present invention there is provided an assay for proAMH in a biological sample comprising proAMH and AMH_{N,C} obtained from a subject, the assay comprising measuring the level of proAMH in the sample using a first binding agent, a second binding agent and a splitting reagent, wherein:
(1) the splitting reagent splits the AMH_{N,C} complex into AMH_{N} and AMH_{C}; and
(ii) when the first binding agent binds to the N-terminal domain of proAMH and AMH_{N,C}, the second binding agent is specific for and binds to the C-terminal domain of proAMH; or
(iii) when the first binding agent binds to the C-terminal domain of proAMH and AMH_{N,C}, the second binding agent is specific for and binds to the N-terminal domain of proAMH.

In one embodiment, the proAMH assay according to this aspect of the present invention further comprises:
(i) contacting the biological sample with the first binding agent such that it binds to proAMH and AMH_{N,C};
(ii) contacting the bound proAMH and AMH_{N,C} with the splitting reagent in an amount sufficient to split the AMH_{N,C} complex into AMH_{N} and AMH_{C}; and
(iii) contacting the bound proAMH and AMH_{N} or AMH_{C} with the second binding agent and measuring the level of bound proAMH.

In another embodiment, the proAMH assay according to this aspect of the present invention further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound proAMH.

In a further aspect of the present invention there is provided an assay for AMH_{N,C} in a biological sample comprising proAMH and AMH_{N,C} obtained from a subject, the assay comprising measuring the level of AMH_{N,C} in the sample using a first binding agent, a second binding agent and a splitting reagent, wherein the first binding agent binds to proAMH and AMH_{N,C}, the second binding agent binds to AMH_{N} or AMH_{C} but does not bind to proAMH or AMH_{N,C}, and wherein the splitting reagent is sufficient to spilt the AMH_{N,C} complex into AMH_{N} and AMH_{C}.

In one embodiment, the AMH_{N,C} assay further comprises:
(i) contacting the biological sample with the first binding agent such that it binds to proAMH and AMH_{N,C};
(ii) contacting the bound proAMH and AMH_{N,C} with the splitting reagent in an amount sufficient to spilt the AMH_{N,C} complex into AMH_{N} and AMH_{C}; and
(ii) contacting the bound proAMH and AMH_{N} or AMH_{C} with the second binding agent and measuring the level of bound AMH_{N} or AMH_{C},
   wherein the level of bound AMH_{N} or AMH_{C} corresponds to the amount of AMH_{N,C} in the sample.

In another embodiment, the AMH_{N,C} assay further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound AMH_{N} or AMH_{C},
   wherein the level of bound AMH_{N} or AMH_{C} corresponds to the amount of AMH_{N,C} in the sample.

The binding agents and splitting reagent used in the assays described herein may also be used in methods for measuring the level of AMH in a biological sample, and in particular measuring the levels of proAMH and AMH_{N,C} In a sample.

Accordingly, in another aspect of the present invention there is provided a method for measuring the level of proAMH in a subject, the method comprising:
(i) obtaining a biological sample from the subject; and
(ii) measuring the level of proAMH in the sample using a first binding agent, a second binding agent and a splitting reagent.

In one embodiment of the method for measuring proAMH in the subject, the splitting reagent splits the AMH_{N,C} complex into AMH_{N} and AMH_{C}, and wherein when the first binding agent binds to the N-terminal domain of proAMH and AMH_{N,C}, the second binding agent is specific for and binds to the C-terminal domain of proAMH, or wherein when the first binding agent binds to the C-terminal domain of proAMH and AMH_{N,C}, the second binding agent is specific for and binds to the N-terminal domain of proAMH.

In another embodiment of the method for measuring proAMH in the subject, the method further comprises:
(i) contacting the biological sample with the first binding agent such that it binds to proAMH and AMH_{N,C};
(ii) contacting the bound proAMH and AMH_{N,C} with the splitting reagent in an amount sufficient to split the AMH_{N,C} complex into AMH_{N} and AMH_{C}; and
(iii) contacting the bound proAMH and AMH_{N} or AMH_{C} with the second binding agent and measuring the level of bound proAMH.

In an alternate embodiment, the method for measuring proAMH according to this aspect of the present invention further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound proAMH.

In another aspect of the present invention there is provided a method for measuring the level of AMH_{N,C} In a subject, the method comprising:
(i) obtaining a biological sample from the subject; and
(ii) measuring the level of AMH_{N,C} In the sample using a first binding agent, a second binding agent and a splitting reagent.

In one embodiment of the method for measuring AMH_{N,C} in the subject, the first binding agent binds to proAMH and AMH_{N,C}, the second binding agent binds to AMH_{N} or AMH_{C} but does not bind to proAMH or AMH_{N,C}, and wherein the splitting reagent is sufficient to split the AMH_{N,C} complex into AMH_{N} and AMH_{C}.

In another embodiment of the method for measuring AMH_{N,C} in the subject, the method further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound AMH_{N} or AMH_{C},

wherein the level of bound AMH_{N} or AMH_{C} corresponds to the amount of AMH_{N,C} in the sample.

In certain embodiments of the proAMH and AMH_{N,C} assays and methods according to the present invention, the first binding agent is immobilised on a solid support.

In other embodiments of the proAMH and AMH_{N,C} assays and methods according to the present invention, the second binding agent comprises a detectable label.

In further embodiments, the first binding agent is an antibody or an antigen binding fragment thereof and/or the second binding agent is an antibody or an antigen binding fragment thereof. The antibody or antigen binding fragment may be selected from the group consisting of a polyclonal, monoclonal, bispecific, chimeric and humanized antibody or an antigen binding fragment thereof, and in certain embodiments the antibody is a monoclonal antibody or antigen binding fragment thereof.

In another embodiment of the AMH_{N,C} assays and methods according to the present invention, the second binding agent comprises a labelled form of AMH_{N} or AMH_{C}, depending on the configuration of the assay or method (i.e. specificity of the first binding agent for the N- or C-terminal domain of AMH).

In yet another embodiment of the AMH_{N,C} assays and methods, the second binding agent is an antibody comprising a detectable label which binds to AMH_{C} or AMH_{N}. For example, Goat anti-rat AMH/MIS antibody (R&D systems, Catalogue# AF1446) that specifically binds to AMH_{C}.

In yet a further embodiment of the proAMH and AMH_{N,C} assays and methods according to the present invention, the splitting reagent is selected from the group consisting of a salt, a detergent, and a chemical reagent sufficient to alter pH. In one embodiment, the splitting reagent comprises deoxycholate.

In yet another embodiment, the proAMH and AMH_{N,C} assays and methods according to the present invention are configured in a format selected from the group consisting of an enzyme linked immunosorbent assay (ELISA), a radiolmmunoassay (RIA), a fluorolmmunoassay, an Immunofluorometric assay, and an immunoradiometric assay.

The invention further provides assays and methods for measuring proAMH comprising binding members that are specific for proAMH.

Accordingly, in another aspect of the present invention there is provided an assay for proAMH in a biological sample comprising proAMH and AMH_{N,C} obtained from a subject, the assay comprising measuring the level of proAMH in the sample using a binding agent specific for proAMH.

In one embodiment, the proAMH assay according to this aspect of the present invention comprises:
(i) contacting the biological sample with the binding agent such that it binds to proAMH; and
(ii) measuring the level of bound proAMH in the sample.

In a related aspect, the present invention also provides a method for measuring proAMH in a subject, the method comprising measuring the level of proAMH in a biological sample obtained from the subject using a binding member specific for proAMH.

In one embodiment of the proAMH specific assay and method according to the present invention, the binding agent specific for proAMH binds to an epitope that spans the cleavage site between the N- and C-terminal domains of proAMH.

In yet a further aspect, the present invention provides an assay for AMH_{N,C} in a biological sample comprising proAMH and AMH_{N,C} obtained from a subject, the assay comprising:
(i) measuring the amount of total AMH in the sample; and
(ii) measuring the amount of proAMH in the sample
   wherein the amount of AMH_{N,C} in the sample represents the difference between total AMH and proAMH measured in the sample.

In a related aspect, the present invention also provides a method for measuring the amount of AMH_{N,C} in a subject, the method comprising:
(i) obtaining a biological sample from the subject;
(ii) measuring the amount of total AMH in the sample; and
(iii) measuring the amount of proAMH in the sample

wherein the amount of AMH_{N,C} In the sample represents the difference between total AMH and proAMH measured in the sample.

In yet another aspect of the present invention there is provided a method for measuring the level of proAMH in a subject, the method comprising:
(i) obtaining a biological sample from the subject;
(ii) measuring the level of proAMH in the sample using a binding agent specific for proAMH.

In one embodiment, the amount of AMH in the sample is determined using a conventional assay for AMH.

In another embodiment, the amount of proAMH in the sample is determined using a proAMH assay described herein.

In yet another aspect, the present invention provides an assay for measuring total AMH in a biological sample comprising proAMH and AMH_{N,C}, the assay comprising:
(i) measuring the amount of proAMH in the sample; and
(ii) measuring the amount of AMH_{N,C} in the sample
   wherein the total amount of AMH in the sample represents the sum of the amounts of proAMH and AMH_{N,C} measured in the sample.

In a related aspect, the present invention also provides a method for measuring total AMH in a biological sample comprising proAMH and AMH_{N,C}, the method comprising:
(i) obtaining a biological sample from the subject;
(ii) measuring the amount of proAMH in the sample; and
(iii) measuring the amount of AMH_{N,C} in the sample
   wherein the total amount of AMH in the sample represents the sum of the amounts of proAMH and AMH_{N,C} measured in the sample.

In one embodiment, the amount of proAMH in the sample is determined using an assay or method described herein.

In another embodiment, the amount of AMH_{N,C} In the sample is determined using an assay or method described herein.

In yet another aspect of the present invention there is provided a dual purpose assay for measuring different species of AMH in a biological sample, the dual purpose assay comprising any combination of the assays described herein. In one embodiment, the dual purpose assay measures proAMH and AMH_{N,C}.

In a related aspect, the present invention also provides a method for measuring proAMH and AMH_{N,C} In a subject, the method comprising any combination of the methods for measuring proAMH and AMH_{N,C} described herein.

In related aspects, the present invention also provides kits comprising binding agents, splitting reagents and instructions for performing the assays and methods described herein.

Accordingly, in yet a further aspect of the present invention there is provided a kit for measuring proAMH in a biological sample obtained from a subject comprising proAMH and AMH_{N,C}, the kit comprising a first binding agent, a second binding agent, a splitting reagent and Instructions for how to measure the level of proAMH in the sample.

According to this aspect of the present invention, the components of the kit are further characterised:
(i) the splitting reagent is capable of splitting the AMH_{N,C} complex into AMH_{N} and AMH_{C}; and
(ii) when the first binding agent binds to the N-terminal domain of proAMH and AMH_{N,C}, the second binding agent is specific for and binds to the C-terminal domain of proAMH; or
(iii) when the first binding agent binds to the C-terminal domain of proAMH and AMH_{N,C}, the second binding agent is specific for and binds to the N-terminal domain of proAMH.

In yet another aspect of the present invention there is provided a kit for measuring AMH_{N,C} in a biological sample comprising proAMH and AMH_{N,C} obtained from a subject, the kit comprising a first binding agent, a second binding agent, a splitting reagent, and instructions for how to measure the level of AMH_{N,C} In the sample.

According to this aspect of the present invention, the components of the kit are further characterised:
(i) the splitting reagent splits the AMH_{N,C} complex into AMH_{N} and AMH_{C}; and
(ii) when the first binding agent binds to proAMH and AMH_{N,C}, the second binding agent is specific for and binds to AMH_{N} or AMH_{C}.

In certain embodiments of the kits of the present invention, the first binding agent is immobilised on a solid substrate.

In other embodiments of the kits of the present invention, the second binding agent comprises a detectable label.

In further embodiments, the first binding agent is an antibody or an antigen binding fragment thereof and/or the second binding agent is an antibody or an antigen binding fragment thereof. The antibody or antigen binding fragment may be selected from the group consisting of a polyclonal, monoclonal, bispecific, chimeric and humanized antibody or an antigen binding fragment thereof, and in certain embodiments the antibody is a monoclonal antibody or antigen binding fragment thereof.

In another embodiment of the kit for measuring AMH_{N,C} according to the present invention, the second binding agent comprises a labelled form of AMH_{N} or AMH_{C}, depending on the configuration of the assay (i.e. specificity of the first binding agent for the N- or C-terminal domain of AMH).

In other embodiments of the kit for measuring AMH_{N,C}, the second binding agent is an antibody comprising a detectable label which binds to AMH_{C} or AMH_{N}, for example, Goat anti-rat AMH/MIS antibody (R&D systems, Catalogue# AF1446) which specifically binds to AMH_{C}.

In yet other embodiments of kits for measuring proAMH and AMH_{N,C} according to the present invention, the splitting reagent is selected from the group consisting of a salt, a detergent, and a chemical reagent sufficient to alter pH. In one embodiment, the splitting reagent comprises deoxycholate.

In yet another aspect of the present invention there is provided a kit for measuring proAMH in a biological sample obtained from a subject comprising proAMH and AMH_{N,C}, the kit comprising a binding agent for proAMH together with instructions for how to measure the level of proAMH in the sample.

In one embodiment of the kit for measuring proAMH according to this aspect of the present invention, the binding agent is capable of binding to an epitope that spans the cleavage site between the N- and C-terminal domains of proAMH. The binding agent may be an antibody or antigen binding fragment thereof, and in particular a monoclonal antibody or antigen binding fragment thereof. Further, the binding agent may comprise a detectable label.

These and other aspects of the present invention, which are not limited to or by the information in this Brief Summary of the Invention, are described in more detail below.

### BRIEF DESCRIPTION OF THE FIGURES

FIG. 1 shows a schematic illustration of the various AMH species, Including preAMH, proAMH, AMH_{N,C}, AMH_{N}, AMH_{C}, AMH₂₅₅₋₅₆₀ and AMH₂₅₋₂₅₄ where numbers represent the position in the amino acid sequence with 1 being the initiating codon methionine. The ability of each species to bind to the canonical AMH receptors is indicated.

FIG. 2 shows a schematic illustration of a conventional/commercial ELISA (top panel) and the proAMH ELISA (middle panel) and AMH_{N,C} ELISA (lower panel) according to the present invention. The Illustrated cleavage step involves separation of AMH_{N,C} into AMH_{N} and AMH_{C}. The detection antibody in the lower panel does not bind to proAMH, most commonly because the epitope is masked by the N-terminal domain of proAMH.

FIG. 3 shows detection of proAMH, AMH_{N} and AMH_{C}. Unprocessed or furin-treated recombinant human AMH (rhAMH) was analyzed on western blots under reducing conditions, using antibodies against the N-terminal (panel A) or C-terminal (panel B) domain of AMH. The location of the proAMH, AMH_{N}, AMH_{C} and alternatively cleaved AMH (AMH₂₅₅₋₅₆₀) are marked with arrows. The left lane in each blot shows the molecular weight markers.

FIG. 4 shows the non-specific bands associated with immunoprecipitation. Control goat IgG was added to human blood, and then precipitated. The precipitated proteins were analysed by western blots under reducing conditions, using only a secondary antibody. The expected location of the proAMH, AMH_{N}, AMH_{C} and alternatively cleaved AMH₂₅₅₋₅₆₀ are marked with arrows (see FIG. 3). The left lane shows the molecular weight markers.

FIG. 5 shows proAMH and AMH_{N,C} is present in human blood. AMH was immunopredpitated with N-terminal (N-ip) or C-terminal (C-ip) specific antibodies from plasma samples obtained from a boy (B1) with high AMH levels and a post-menopausal woman (PMW) to act as an AMH-defident control (Table 1). Samples were run on western blot under reducing conditions, and detected with either anti-N terminal (A) or anti-C terminal antibodies (B). The proAMH, AMH_{N} and ANH_{C} bands are indicated by arrows, with the heavy chain and light chain IgG bands indicated by the upper and lower arrowheads, respectively. The molecular weight of AMH₂₅₅₋₅₆₀ is indicated by the double arrowhead. The left lane in each blot shows the molecular weight markers, with the right lane a 5 ng rhAMH_{C} control.

FIG. 6 shows an anti-C terminal antibody selectively immunoprecipitates AMH_{C}. rhAMH (proAMH, with lesser levels of AMH_{N,C}, FIG. 3) or rhAMH_{C} was immunoprecipitated with beads coated with either an anti-C terminal or anti-N terminal antibody of AMH. The precipitates were then analyzed by western blot under reducing conditions, using an anti-C antibody to detect AMH_{C} and proAMH. As expected, anti-C terminal antibody precipitated AMH_{C} (lane 2) but not proAMH (lane 3), whereas the anti-N terminal antibody precipitated proAMH (lane 4). Lanes 5 and 6 show that there is minimal precipitation of recombinant forms of AMH when the beads are not coated with AMH-specific antibodies. The strong bands indicated by arrowheads are the antibodies used for precipitation. The left lane (lane 1) shows the molecular weight markers.

FIG. 7 shows proAMH and AMH_{N,C} Is present in the blood of men, women and boys. AMH was immunoprecipitated with an anti-N-terminal antibody and the western blot was probed with both the C-terminal and N-terminal antibodies. Plasma samples were obtained from boys (B2-3), adult men (M1-3), pre-menopausal women (W1-3) and one post-menopausal woman (PMW). The proAMH, AMH_{N} and AMHe bands are indicated by arrows, with the heavy chain and light chain IgG bands indicated by the upper and lower arrowheads, respectively. The molecular weight of AMH₂₅₅₋₅₆₀ is indicated by the double arrowhead. The high-molecular weight bands are evident in low gain scans (A), with higher gain scan required to detect AMH_{C} (B) (see also FIG. 8). The left hand lane shows the molecular weight markers.

FIG. 8 shows AMH_{N,C} is present in the blood of men and women. AMH_{C} precipitated from the blood of adults is near the detection limit of a western. The samples from FIG. 7 were therefore run a second time to confirm the presence of AMH_{C}. The original scan is illustrated in (A), and was quantified by densitometry (C). The original image was then enhanced linearly to give visual confirmation of the densitometry measurements (B).

FIG. 9 shows deglycosylation of AMH was conducted on AMH immunoprecipitated from a boy with a combination of 5 deglycosylating enzymes (PNGase F, Slalldase A, O-glycosidase, β(1→4)-galactosidase and β-N-acetylglcuosaminidase). The AMH immunoprecipitated from the plasma of boy B3 was incubated with or without deglycosylation enzymes for 3 hours at 37° C. and detected by western blot with the anti-N-terminal AMH antibody. The immunoprecipitating antibody was covalently linked to the agarose beads to minimise any overlap between the IgG heavy chain and deglycosylated AMH_{N}. The thick arrows indicate the position of the bands for proAMH and AMH_{N} and the arrowhead identifies the Immunoprecipitating IgG heavy chain before deglycosylation treatment. Thin arrows indicate the new location of the proAMH and AMH_{N} bands after treatment with deglycosylating enzymes. The left lane in each blot shows the molecular weight markers.

FIG. 10 shows AMH is glycosylated in adult samples. Deglycosylation of AMH was conducted on AMH immunoprecipitated from two men (M4 & M5) and two pre-menopausal women (W5 & W5). A combination of deglycosylating enzymes was used; PNGase F, Sialidase A, O-glycosidase, β(1→4)-galactosidase (β(1→4)-Gal-ase) and β-N-acetylglcuosaminidase (β-GlcNAc-ase). The bands were detected on reducing conditions western blot with the anti-N-terminal AMH antibody which resolved faint bands. M5 showed the strongest signal with a visible proAMH band at 72 kDa and two AMH_{N} bands at ˜60 kDa and both bands shifted after deglycosylation. W4 and W5 do not show bands before deglycosylation but do have faint proAMH and AMH bands after deglycosylation. This is consistent with the sample from M5 and the boys in FIG. 9, where the anti-N-terminal antibody appears to have a higher affinity for deglycosylated proAMH and AMH_{N}.

FIG. 11 shows proAMH binds to the ELISA matrix of a commercial AMH assay (Beckman Coulter; Catalogue #A79765). rhAMH_{C} (c), rhAMH_{N} (n) or rhAMH (AMH) were loaded onto the matrix from an AMH ELISA and onto a matrix from an Inhibin B (InhB) ELISA (Beckman Coulter; Catalogue #A81303). The proteins captured by the ELISAs were then recovered and analyzed by western blots using either an anti-N terminal antibody or an anti-C terminal antibody. rhAMH_{C}, rhAMH_{N} and rhAMH were also run directly on the western, to act as standards. The proAMH, AMH_{C} and AMH_{N} bands are indicated. A non-specific band of variable intensity was recovered from the AMH ELISA wells, and is indicated by arrowheads. The left hand lane of blot shows the molecular weight markers.

FIG. 12 shows proAMH from human blood binds to the ELISA matrix of a commercial AMH assay (Beckman Coulter; Catalogue #A79765). Blood from 3 boys (B4-B6) were mixed with the commercial assay buffer and added to either AMH or Inhibin B (InhB) wells. The captured proteins were recovered and analyzed by western blots, using antibodies to the C-terminal and N-terminal. The non-specific band that is variably recovered from the wells is indicated by an arrowhead (see also FIG. 11). The left hand lane of each blot shows the molecular weight markers.

FIG. 13 shows that deoxycholate can prevent AMH_{N} from being captured. Varying concentrations of dexoycholate was added to samples of rhAMH or rhAMH that had been treated with furln to produce rhAMH_{N,C}. After incubation, the samples were added to ELISA wells from a commercial assay (Beckman Coulter; Catalogue #A79765), and the captured proteins analyzed by western blots. The left hand lane shows the molecular weight markers.

FIG. 14 shows that deoxycholate can release AMH_{N} captured from blood. AMH was captured from the serum, and then washed with either phosphate buffer (PB) or 0.2% sodium deoxycholate. The serum in all 6 wells was from the same adult. The captured proteins were recovered and analyzed by western blots. The two columns on the right hand side of the gel did not have serum added.

FIG. 15 shows an Illustration of the AMH used in the proAMH assay in FIG. 16. rhAMH was incubated with furin and an aliquot of the original rhAMH and the furin-treated AMH analyzed by western blots, using the anti-N terminal antibody. The rhAMH is predominantly proAMH, whereas the furin-treated rhAMH is heavily enriched AMH_{N,C}, with minor levels of residual proAMH present.

FIG. 16 shows demonstration of a proAMH-specific ELISA. Varying concentrations of rhAMH (proAMH) and furin-treated rhAMH (AMH_{N,C}) were assayed using a commercial assay (Beckman Coulter, Catalogue #A79765), with the added deoxycholate step.

FIG. 17 shows capture of proAMH and AMH_{N,C} using another commercial ELISA assay matrix (AnSH Labs AMH ELISA; Catalogue #AL-105-1). rhAMH_{C}, rhAMH_{N}, rhAMH or human serum were loaded onto wells from an Ansh Labs AnshLite Ultrasensitive AMH/MIS chemlluniescent immunoassay. The proteins captured by the wells were then recovered an anlayzed by western blots using either an anti-N terminal antibody or an anti-C terminal antibody. The proAMH, AMH_{C}, AMH_{N} and AMH₂₅₅₋₅₆₀ bands are indicated. A non-specific band of variable intensity was recovered from the wells, and is indicated by arrowheads. The left hand lane of each blot shows the molecular weight markers. The serum sample of each blot was initially separated by one lane (deleted) from the recombinant proteins.

FIG. 18 shows that proAMH ELISA detects AMH from human samples and there is no difference in detection compared to the rhproAMH initially used as a standard.

FIG. 19 shows detection of various forms of rh-AMH at 14 nM by the duoset ELISA (R&D systems, catalogue #DY1737) under normal running conditions (control) or when samples have been pre-treated with deoxycholate. Under normal conditions the ELISA detects AMH_{C} but not proAMH and only a small signal is given for AMH_{N,C}. Pre-treatment of AMH_{N,C} with deoxycholate increases the signal to a level equivalent to AMH_{C} but only has a minor effect on the proAMH preparation which contains small quantities of AMH_{N,C}.

FIG. 20 shows recovery of various forms of AMH at 14 nM from the duoset ELISA (R&D systems, catalogue #DY1737) under normal running conditions (control) or when samples have been pre-treated with deoxycholate. AMH_{N,C} does not bind to the ELISA capture antibody as efficiently as AMH_{C} under normal conditions. Deoxycholate pre-treatment, causes the amount of AMH_{C}-binding in rh-AMH_{N,C} samples relative to the amount of purified AMH_{C} binding. No appreciable binding of proAMH or co-capture of AMHn as part of an AMH_{N,C} complex was observed under normal conditions or with deoxycholate pre-treatment (for examples of proAMH and AMH_{N,C} binding to ELISAs, FIGS. 12, 13 & 14). This indicates that the small signal from the deoxycholate-treated proAMH sample in FIG. 19 is due to the presence of low levels of AMH_{N,C} in the proAMH preparation.

FIG. 21 shows demonstration of an ELISA that detects AMH_{C} or AMH_{N,C} but not proAMH. Varying concentrations of non-treated rhAMH (proAMH with small quantities of AMH_{N,C}) and furin-treated rhAMH (AMH_{N,C} with small quantities of proAMH) were assayed using a commercial assay for AMH_{C} (R&D systems, catalogue #DY1737), with a deoxycholate pre-treatment step. Rh-AMH_{C} (R&D systems, catalogue #1737-MS) was assayed to demonstrate that deoxycholate does not interfere with normal function of the assay.

FIG. 22 shows a sequence alignment between human, mouse, rat and bovine AMH sequences.

FIG. 23 shows total AMH concentration plotted against % proAMH in boys (A), men aged 24-65 (B) and men older than 65 years of age (C).

FIG. 24 shows Total AMH and % proAMH values in females. (A) Total AMH changes vs. age; (B) % proAMH vs. age and (C) % proAMH vs total AMH with fitted regression lined for girls aged 4-11 (solid circles, n=17), women aged 19-31 (solid diamonds, n=31) and women aged 31-46 (open circles, n=13).

### DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the Invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### Molecular Definition of AMH

Anti-Müllerian Hormone (AMH) is synthesised as a glycosylated cysteine-linked homodimer of the 560 amino add prepropeptide (preAMH). The first 24 amino acids are removed during synthesis yielding the prohormone (proAMH), which does not activate AMH receptors, which includes, for example, Type II AMH Receptor (AMHRII). Enzymatic cleavage at a monobasic recognition site between amino acids 451 and 452 yields a 25 kDa C-terminal dimer (AMH_{C}) and 120 kDa N-terminal dimer (AMH_{N}), which initially remain associated in a non-colavent complex (AMH_{N,C}). AMH_{C} is the receptor-activating component of AMH. Some recombinant forms of AMH also contain alternatively cleaved AMH, which yields an extended C-terminal peptide consisting of amino adds 255-560 (AMH₂₅₅₋₅₆₀). It is unclear whether this form of AMH binds to or activates AMH receptors.

The nomenclature used to describe AMH species is variable in the literature, and in product descriptions of AMH-related products. In some instances, it is unclear which form of AMH has been studied. AMH_{N,C} and AMH_{N} have both been described as proAMH, as they are a precursor forms of AMH_{C}. AMH_{N,C} and AMH_{C} have both been referred to as mature AMH.

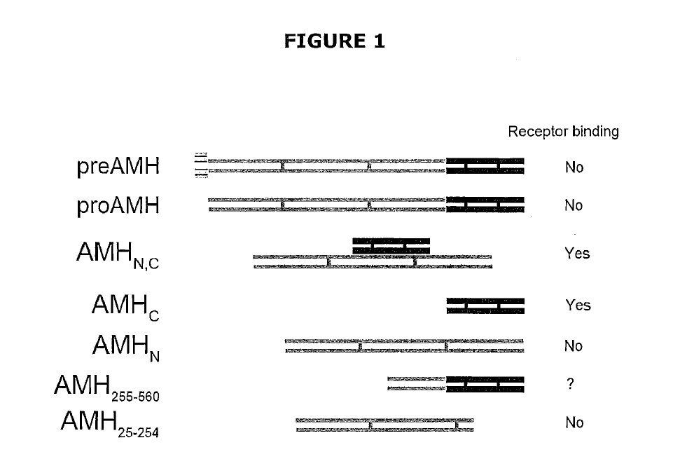

In referring to the different species of AMH used in this specification, FIG. 1 is particularly instructive. For further context:

“preAMH” is intended to mean the large precursor or prepropeptide of AMH which includes a short signal sequence (24 amino adds) followed by the propeptide that forms homodimers.

“proAMH” is intended to mean the prohormone that corresponds to a 140 kDa dimer of identical disilflde-linked 70 kDa monomer subunits; each monomer contains an N-terminal domain and a C-terminal domain.

“N-terminal domain of AMH” when used in the context of a human AMH sequence is intended to mean residues 1-451 of the human prepropeptide or residues 25-451 if the leader sequence has been removed

“C-terminal domain of AMH” when used in the context of a human AMH sequence is intended to comprise residues 452-560 the human propeptide.

“AMH_{N}” is intended to mean a dimer of identical N-terminal domains.

“AMH_{C}” is intended to mean a dimer of identical C-terminal domains.

“AMH_{N,C}” is intended to mean a non-covalent complex consisting of a 120 kDa N-terminal dimer (AMH_{N}) and a 25 kDa C-terminal dimer.

“AMH₂₅₅-so” is intended to mean an alternatively cleaved form of AMH which consists in an extended C-terminal peptide comprising amino adds 255-560.

When used throughout this specification, and unless defined otherwise, these terms should be taken to have the meanings referred to above.

### OTHER DEFINITIONS

The following terms have the following meanings when used herein.

The term “antibody” refers to an immunoglobulin molecule capable of specifically binding an antigen, such as, for example, AMH, and typically by binding an epitope or antigenic determinant of AMH, such as, for example, a C-terminal or N-terminal region of AMH, including AMH_{N} and AMH_{C}. As used herein, the term “antibody” broadly includes full length antibodies and antigen binding fragments or regions thereof. Also included are monoclonal and polyclonal antibodies, multivalent and monovalent antibodies, multispecific antibodies (for example bi-specific antibodies), chimeric antibodies, human antibodies, humanized antibodies and antibodies that have been affinity matured. An antibody binds selectively or specifically to a region or domain, such as the N- or C-terminal region of AMH, if the antibody binds preferentially to a region or domain of AMH which has, e.g. has less than 25%, or less than 10%, or less than 1% or less than 0.1% cross-reactivity with non-AMH antigens/epitopes or other non-target AMH species, when appropriate. Usually, the antibody will have a binding affinity (dissociation constant (Kd) value), for the antigen or epitope of about 10⁻⁶, or 10⁻⁷M, 10⁻⁸M, or 10⁻⁹M, or 10⁻¹⁰, or 10⁻¹¹ or 10⁻¹²M. Binding affinity may be assessed using surface plasma resonance, for example, or Scatchard analysis.

As used herein, an “antigen binding fragment” or “antibody fragment” or “binding fragment” when used in reference to an antibody, means a portion of the Intact antibody that preferably retains most or all, or minimally at least one of, the normal binding functions of the intact antibody. Examples of antibody fragments include Fab, Fab′, F(ab′)₂ and Fv fragments, linear antibodies, diabodies, single chain antibodies (ScFV), domain antibodies and multispecific antibodies.

As used herein, the term “N-terminal antibody” when used in the context of AMH, means an antibody (or antigen binding fragment, antibody fragment or binding fragment) raised against the N-terminal domain of AMH (defined by amino add residues 1-451 of the human propeptide) which specifically binds to an epitope or antigenic determinant of the N-terminal region of AMH.

The terms “N-terminal antibody” and “anti-N terminal antibody” may be used interchangeably throughout this specification and are intended to have the same meaning.

As used herein, the term “C-terminal antibody” when used in the context of AMH, means an antibody (or antigen binding fragment, antibody fragment or binding fragment) raised against the C-terminal domain of AMH (defined by amino acid residues 452-560 of the human propeptide) which specifically binds to an epitope or antigenic determinant of the C-terminal region of AMH.

The terms “C-terminal antibody” and “anti-C terminal antibody” may be used interchangeably throughout this specification and are intended to have the same meaning.

As used herein, the terms “domain” and “region” when used in the context of a domain or region of a protein, may be used interchangeably. For example, in referring to the Examples and experiments which follow, antibodies raised against the “N-terminal domain” or “N-terminal region” of AMH could be taken to have the same intended meaning. However, this is not always the case. By way of further illustration, the term “N-terminal antibody” could be understood to mean an antibody (or antigen binding fragment, antibody fragment or binding fragment) which has been raised against a region, such as an epitope or antigenic determinant, of the N-terminal domain of AMH.

The term “epitope” Includes any antigenic (e.g., a protein) determinant capable of specific binding to an antibody and/or a T cell receptor. That is, a site on an antigen to which B and/or T cells respond. Epitopic determinants usually consist of chemically active surface groupings of molecules such as amino acids or sugar side chains, and usually have specific three-dimensional structural characteristics, as well as specific charge characteristics. An epitope typically includes at least 3, 5 or 8-10 amino acids. The amino acids may be contiguous, or non-contiguous amino acids juxtaposed by tertiary folding. Conformational and non-conformational epitopes are distinguished in that the binding to the former but not the latter is lost in the presence of denaturing solvents.

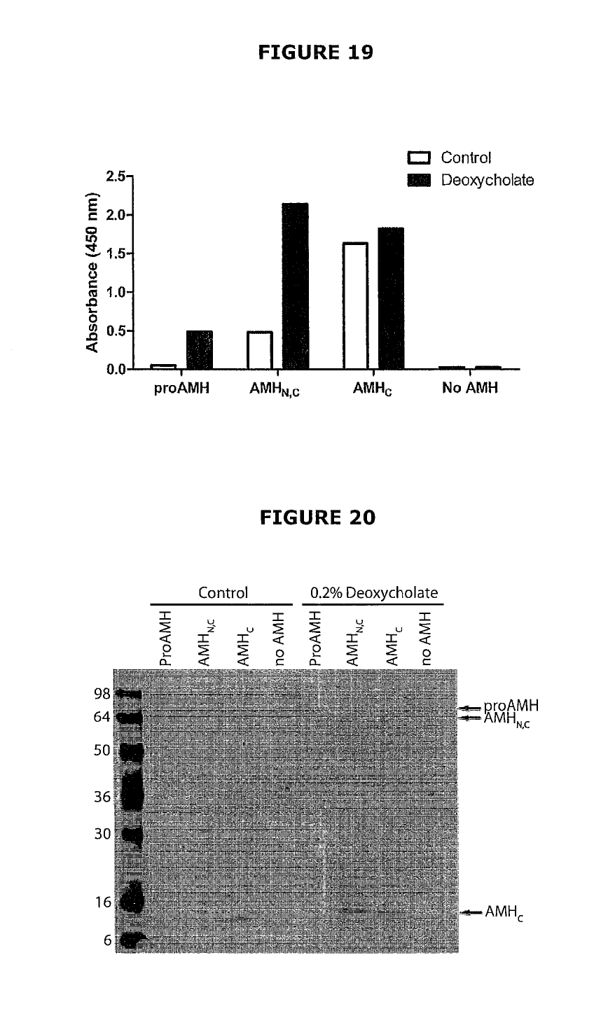

As used herein, the term “antigenic variant” refers to polypeptide sequences different from the specifically identified sequences, wherein one or more amino acid residues are deleted, substituted, or added. Substitutions, additions or deletions of 1, 2, 3, 4, 5, 6, 10, 15, 20, 40, 50 or more amino adds are specifically contemplated. Variants may be naturally-occurring allelic antigenic variants, or non-naturally occurring antigenic variants. Variants may be from the same or from other species and may encompass homologues, paralogues and orthologues. In certain embodiments, antigenic variants of the polypeptides useful in the invention have biological activities including hormone function or antigenic-binding properties that are the same or similar to those of the parent polypeptides. The term “antigenic variant” with reference to (poly)peptides encompasses all forms of polypeptides as defined herein. The term “antigenic variant” encompasses naturally occurring, recombinantly and synthetically produced polypeptides. For example, an antigenic variant of human AMH may include the non-human sequences of AMH, such as those AMH sequences derived from mouse, rat, sheep, bovine, pig etc (see, for example, FIG. 19).

In addition to computer/database methods known in the art, polypeptide antigenic variants may be identified by physical methods known in the art, for example, by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

An “Isolated antibody” is an identified antibody that has been separated or recovered, or both, from a component of its natural environment. For example, separated from proteins including enzymes and hormones. In one embodiment, the antibody is purified to at least 95%, or 96% or 97% or 98% or 99% by weight of antibody. Purity can be determined by the Lowry method, for example. Ordinarily the antibody will be prepared by at least one purification step.

As used herein, a “monoclonal antibody” means an antibody that is a highly specific antibody directed against (or which binds to) a single antigen target. A monoclonal antibody may be obtained from a population of homogenous or substantially homogenous antibodies wherein each monoclonal antibody is identical and/or bind the same epitope, except for natural mutations that may occur in minor amounts. Monoclonal antibodies are prepared using methods known the art, such as, for example, in Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. (Jointly and individually referred to herein as Harlow and Lane).

As used herein, a “polydonal antibody” means an antibody which may be directed against (or which may bind to) multiple antigen targets. Polyclonal antibodies are prepared using methods known the art (such as, for example, in Harlow and Lane, ibid).

The term “binding agent” as used herein refers to any solid or non-solid material capable of binding a species of AMH, fragment or an antigenic variant thereof. In one embodiment the term refers to any natural or non-natural molecule that binds to a species of AMH, fragment or antigenic variant thereof. Examples of binding agents include proteins, peptides, nucleic acids, carbohydrates, lipids, and small molecule compounds. One selective or specific binding agent is an antibody or antigen binding fragment thereof.

The term “sample” or “biological sample” as used herein means any sample taken or derived from a subject. Such a sample may be obtained from a subject, or may be obtained from biological materials intended to be provided to the subject. For example, a sample may be obtained from blood being assessed, for example, for ovarian reserve in a subject, and used to evaluate blood for the levels of Anti-Müllerian Hormone (AMH). Included are samples taken or derived from any subjects such as from normal healthy subjects and/or healthy subjects for whom it is useful to understand the levels of AMH. Preferred samples are body fluid samples. The term “body fluid sample” as used herein refers to a sample of bodily fluid obtained for the purpose of, for example, diagnosis, prognosis, classification or evaluation of a subject of interest, such as a patient. In certain embodiments, such a sample may be obtained for the purpose of determining ovarian reserve. The sample may be any sample known in the art in which AMH can be detected. Included are any body fluids such as a whole blood sample, plasma, serum, ovarian follicular fluid sample, seminal fluid sample, cerebrospinal fluid, saliva, sputum, urine, pleural effusions, interstitial fluid, synovial fluid, lymph, tears, for example, although whole blood sample, plasma, serum, ovarian follicular fluid sample and seminal fluid sample are particularly suited for use in this invention. In addition, one of skill in the art would realise that certain body fluid samples would be more readily analysed following a fractionation or purification procedure, for example, separation of whole blood into serum or plasma components.

The term “purified” as used herein does not require absolute purity. Purified refers in one embodiment to at least 90%, or 95%, or 98%, or 99% homogeneity of, to provide an example, of a polypeptide or antibody in a sample.

The term “subject” as used herein is preferably a mammal and includes human, and non-human mammals such as cats, dogs, horses, cows, sheep, deer, mice, rats, primates (Including gorillas, rhesus monkeys and chimpanzees), possums and other domestic farm or zoo animals. Thus, the assays, methods and kits described herein have application to both human and non-human animals, in particular, and without limitation, humans, primates, farm animals including cattle, sheep, goats, pigs, deer, alpacas, llamas, buffalo, companion and/or pure bred animals including cats, dogs and horses. Preferred subjects are humans, and most preferably “patients” who as used herein refer to living humans who may receive or are receiving medical care or assessment for a disease or condition. Further, while a subject is preferably a living organism, the Invention described herein may be used in post-mortem analysis as well.

The term “ELISA” as used herein means an enzyme linked immunosorbent assay, a type of competitive binding assay comprising antibodies and a detectable label used to quantitate the amount of an analyte in a sample.

The term “capture antibody” as used herein means an antibody which is typically immobilized on a solid support such as a plate, bead or tube, and which antibody binds to and captures analyte(s) of Interest, for example proAMH or AMH_{N,C} or both.

The term “detection antibody” as used herein means an antibody comprising a detectable label that binds to analyte(s) of interest. The label may be detected using routine detection means for a quantitative, semi-quantitative or qualitative measure of the analyte(s) of Interest, for example proAMH or AMH_{N,C} or both.

As used herein, the term “relating to the presence or amount” of an analyte reflects that assay signals are typically related to the presence or amount of an analyte through the use of a standard curve calculated using known concentrations of the analyte of interest. As the term is used herein, an assay is “configured to detect” an analyte If an assay can generate a detectable signal indicative of the presence or amount of a physiologically relevant concentration of the analyte. Typically, an analyte is measured in a sample.

A level “higher” or “lower” than a control, or a “change” or “deviation” from a control (level) in one embodiment is statistically significant. A higher level, lower level, deviation from, or change from a control level or mean or historical control level can be considered to exist If the level differs from the control level by about 5% or more, by about 10% or more, by about 20% or more, or by about 50% or more compared to the control level. Statistically significant may alternatively be calculated as P≦0.05. Higher levels, lower levels, deviation, and changes can also be determined by recourse to assay reference limits or reference intervals. These can be calculated from intuitive assessment or non-parametric methods. Overall, these methods may calculate the 0.025, and 0.975 fractiles as 0.025*(n+1) and 0.975 (n+1). Such methods are well known in the art. Presence of a marker absent in a control may be seen as a higher level, deviation or change. Absence of a marker present in a control may be seen as a lower level, deviation or change.

The term “conventional assay for AMH” as used herein is intended to mean any commercial available assay for measuring (total) AMH available at the filing date, and Includes but is not limited to Beckman Coulter AMH Generation II ELISA Kit (BC ELISA; Catalogue # A79765) and ANSH Labs AMH ELISA (ANSH ELISA; Catalogue #A79765).

### DETAILED DESCRIPTION

Until now, it has been widely assumed that there is only a single molecular form of AMH in blood, namely the bioactive cleaved form, AMH_{N,C}, which does not vary with age or gonadal pathology. However, confounding these customary views, the Applicants now provide the first direct evidence to show that human blood contains a mixture of AMH species, which is predominantly or almost exclusively proAMH and AMH_{N,C}.

The present invention therefore provides assays, methods and kits for the detection and measurement of the different species of AMH present in a biological sample, and in particular a biological sample such as a whole blood sample, plasma or serum.

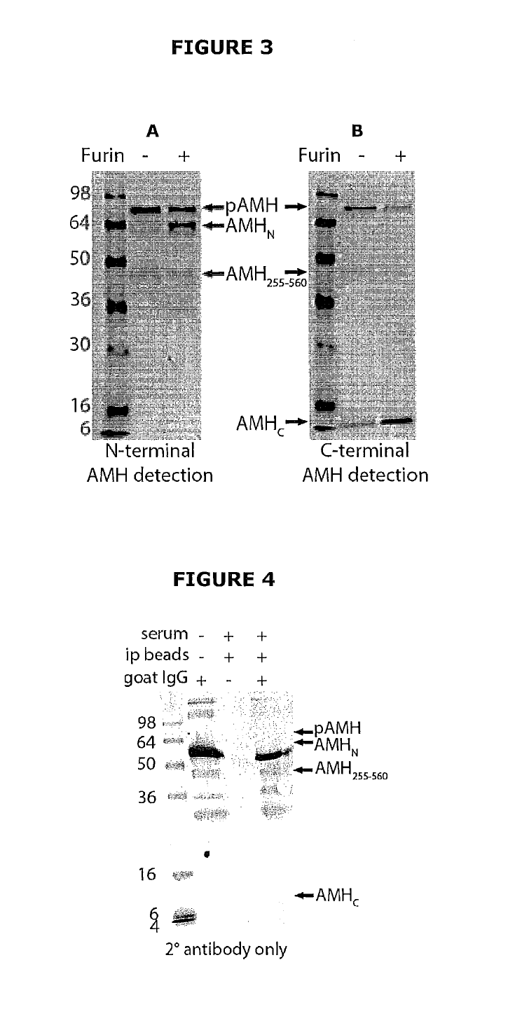

Initially, the Applicants sought to confirm the different species of AMH present for unprocessed and furin (enzyme) treated recombinant human AMH (rhAMH). The molecular weights and Immunoreactivity of proAMH, AMH, AMH_{C} and AMH₂₅₅₋₅₆₀ were investigated by western blot under reducing-conditions using antibodies against the N- and C-terminal regions of AMH (FIG. 3; Example 1).

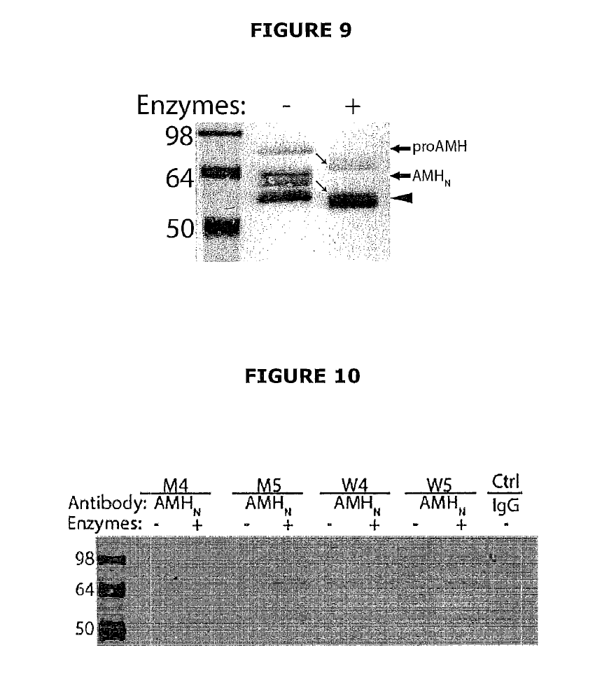

The recombinant human form of AMH (rhAMH) derived from HEK cells was predominantly a 72 kDa species, with lesser amounts of other forms of AMH observed. The apparent molecular weight for proAMH is larger than predicted from its amino acid sequence due to its glycosylation state (FIGS. 9 and 10; Example 5).

The 72 kDa species is designated proAMH because it bound both the C- and N-terminal antibodies, and because pretreatment with the proprotein convertase furin diminished the Intensity of the band. Conversely, the intensity of the AMH_{C} and AMH_{N} bands increased following furin treatment, providing further confirmation of their identities. proAMH was more efficiently detected by the N-terminal than the C-terminal antibody (FIG. 3A versus FIG. 3B; Example 1). AMH_{C} at 12.5 kDa was detected exclusively by the C-terminal antibody (FIG. 3B), whereas no equivalent band was detected using the N-terminal antibody (FIG. 3A)

The N-terminal antibody exclusively bound to two AMH_{N} bands at 62 and 64 kDa (FIG. 3A). AMH is a glycosylated protein, and the two AMH_{N} bands resolve to a single band when AMH is deglycosylated (data not shown).

Similar to proAMH, the apparent molecular weight of the AMH_{N} band is larger than predicted from amino acid sequence, due to its glycosylation.

A lower abundance 47 kDa band was also present. This band contained both N and C-terminal epitopes, with its size being consistent with the previously described AMH₂₅₅₋₅₆₀.

AMH in blood is generally presumed to be AMH_{N,C}, and over 400 scientific papers have been published on this basis. Applicants now provide the first direct evidence to show that blood contains a mixture of AMH species, and predominantly proAMH and AMH_{N,C}.

The level of AMH in blood is too low to be detected by direct western blot analysis, necessitating enrichment of AMH species, by techniques known in the art. In one embodiment of the present invention, enrichment of AMH species from a sample is performed by immunoprecipitation.

Immunoprecipitation is a technique of precipitating a protein antigen out of solution using an antibody that specifically binds to that particular protein. Broadly, this process can be used to isolate and concentrate a particular protein from a sample containing many thousands of different proteins. Immunoprecipitation requires that the antibody be coupled to a solid substrate at some point during the precipitation process, such as, to highly porous sepharose or agarose resins or slurries, or to non/magnetic beads, for example. An artefact of immunoprecipitation is the inclusion of non-specific bands which appear in western blots from the precipitating reagents. Accordingly, the use of this technique necessitates that the non-specific bands do not obscure the bands of the target protein(s) to be analysed.

For immunoprecipitation of AMH, non-specific bands were identified by adding control IgG from a non-immunised goat to human serum. The resulting bands were different in size to proAMH, AMH_{N} and AMH_{C} (compare FIGS. 3 and 4; Example 2). Faint non-specific bands were detected at approximately 47 kDa (FIG. 4). These bands would obscure trace but not significant levels of AMH₂₅₅₋₅₆₀.

Having validated an approach to enrichment of AMH through immunoprecipitation, Applicants then sought to investigate the species of AMH present in blood. It is widely acknowledged, and known to a person skilled in the art, that boys possess higher blood levels of AMH compared to, for example, prepubescent girls, or adult men or women. Accordingly, the Initial analysis of blood AMH was performed using plasma samples obtained from pre-pubescent boys. Since post-menopausal women (PMW) only possess trace levels of AMH (Table 1; Example 4) a plasma sample derived from a PMW subject was used as a negative control.

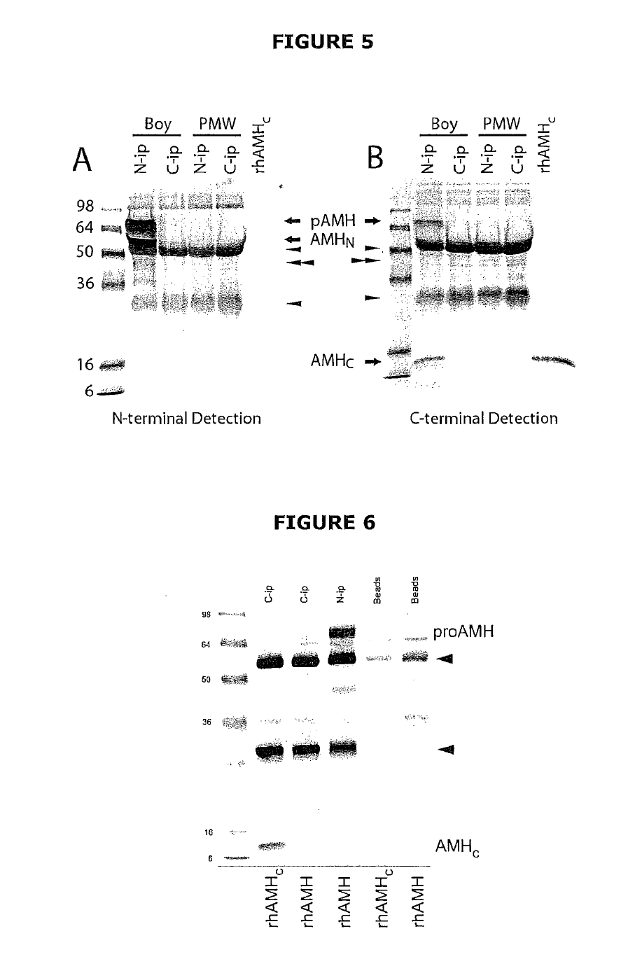

As demonstrated in FIG. 5, significant levels of proAMH and AMH_{N,C} were detected using antibodies to either the N- or C-terminal domains of AMH. The strongest band immunoprecipitated by the N-terminal antibody had the characteristics of proAMH. That is a 72 kDa protein was detected using both N- and C-terminal antibodies to AMH (FIGS. 5A and 5B; Example 3), which was not present in the negative control. The N-terminal antibody also immunoprecipitated bands with molecular weights appropriate for AMH_{N} and AMH_{C}, which again were not present in the negative control. The detection of AMH_{C} suggests it is coimmunoprecipitating as part of the AMH_{N,C} complex because a detection antibody to the N-terminal domain does not directly bind to AMH_{C} (FIG. 5A). Collectively, these data provide the first direct evidence to show that human blood contains a mixture of proAMH and AMH_{N,C}.

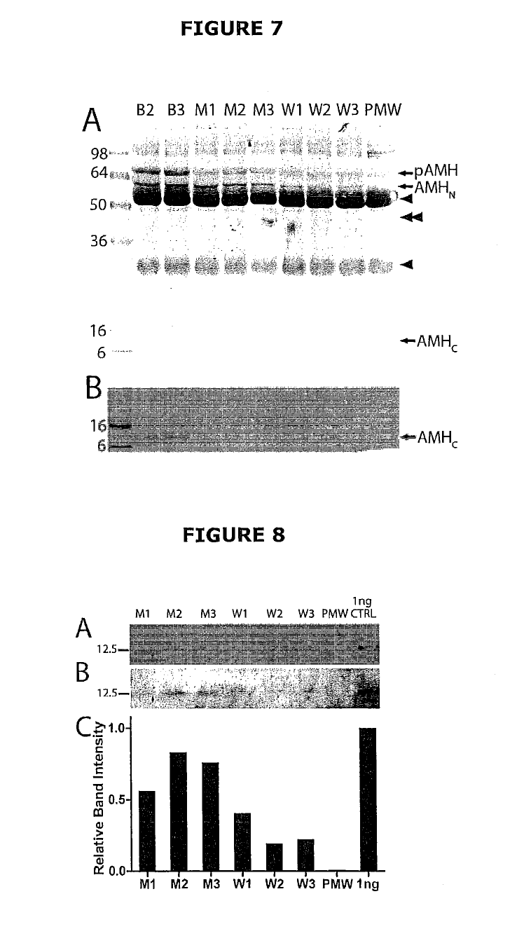

Further investigations conducted by the Applicants then confirmed that proAMH and AMH_{N,C} is present in adult serum from both sexes. AMH was immunoprecipitated (using a N-terminal antibody to AMH) from plasma samples obtained from an additional two pre-pubescent boys (B2, B3), three young men (M1, M2, M3) and three pre-menopausal women (W1, W2, W3), with a post-menopausal woman included as a negative control (PMW; Example 4). As shown in FIG. 7, proAMH and AMH_{N} was detected in the blood of boys, men and women with the Intensity of the bands being broadly commensurate with the estimate of proAMH levels as quantified by ELISA (FIG. 7; Table 1). The PMW control had no detectable levels of AMH_{N} and only trace levels of proAMH (FIG. 7).

With respect to AMH_{N,C}, the N-terminal antibody co-Immunoprecipitated AMH_{C} from the plasma of boys (FIG. 7; lanes B2 and B3), while the levels of AMH_{C} in the adult samples were low (FIGS. 7, 8), as could be expected based on levels quantified by ELISA (Example 4; Table 1). The relative amounts of AMH_{C} were therefore confirmed using densitometry analysis (FIG. 8C). Again, no detectable levels of AMH_{C} were measured for the PMW control (FIG. 8C).

Collectively, these data provide the first direct evidence to show that the blood of men and pre-menopausal women, as well as boys, contains a mixture of AMH species, which is predominantly or exclusively proAMH and AMH_{N,C}. It is important to note that proAMH and AMH_{N,C} are distinct species with different biological functions and activities. For example, AMH_{N,C} activates AMH receptors whereas proAMH does not (Di Clemente et al. (2010) Mol Endocrinol 24:2193-2206). Accordingly, Applicants have now identified a clear need to develop new detection means which measure the different forms of AMH in a sample, such as a biological sample, without interference from other AMH species.

In certain aspects of the present invention, Applicants have developed a novel approach to specifically measure proAMH in a biological sample comprising different forms of AMH, including proAMH and AMH_{N,C}.

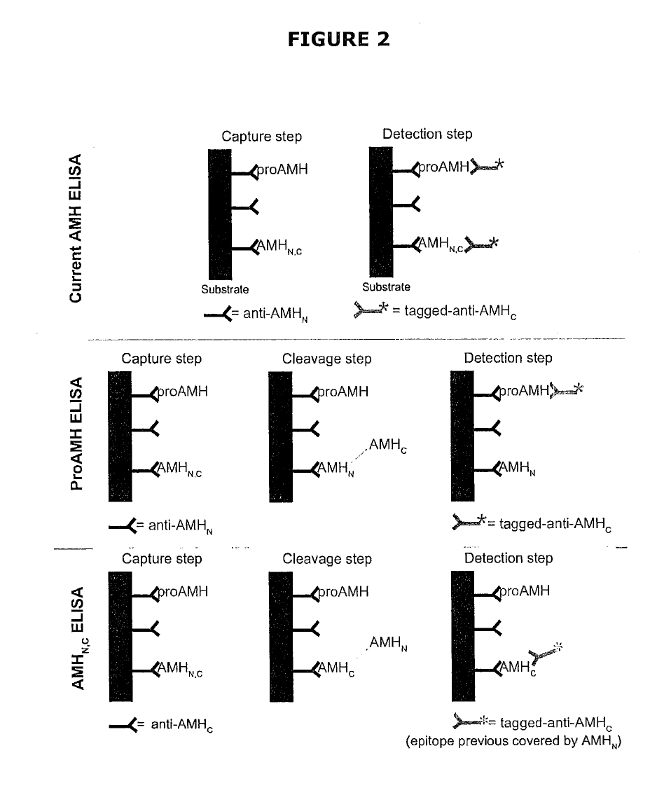

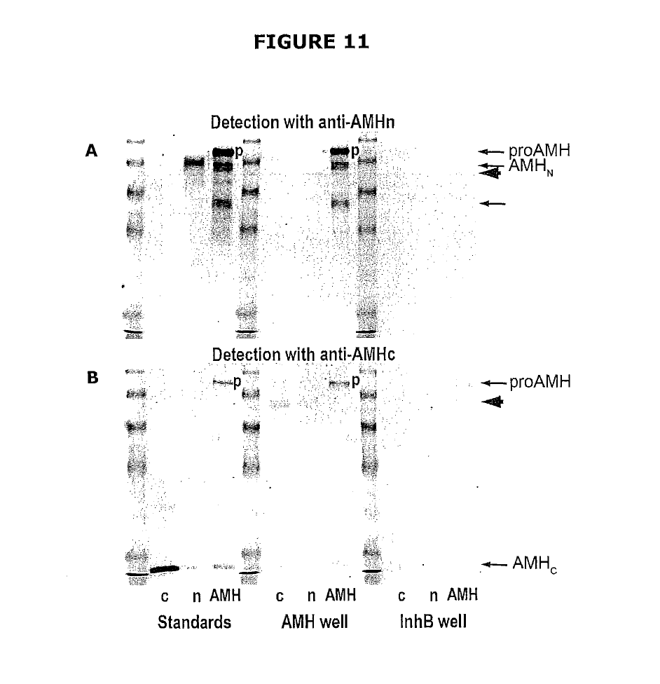

By way of Illustration only, a general concept underlying one aspect of the present invention (i.e. proAMH specific assay) is presented in FIG. 2, contrasted against conventional AMH ELISA assay platforms. Importantly, this schematic shows that conventional ELISAs do not distinguish between proAMH and AMH_{N,C}, in other words conventional ELISAs measure both proAMH and AMH_{N,C}, whereas, in one aspect, the proAMH assay according to the present invention is specific for proAMH. This observation is supported by experimental evidence presented in Examples 6-8 and FIGS. 11-18 which follow.

Accordingly, in one aspect of the present invention there is provided an assay for proAMH in a biological sample comprising proAMH and AMH_{N,C} obtained from a subject, the assay comprising measuring the level of proAMH in the sample using a first binding agent, a second binding agent and a splitting reagent, wherein:
(i) the splitting reagent splits the AMH_{N,C} complex into AMH_{N} and AMH_{C}; and
(ii) when the first binding agent binds to N-terminal domain of AMH, the second binding agent binds to the C-terminal domain of AMH; or
(ii) when the first binding agent binds to the C-terminal domain of AMH, the second binding agent binds to the N-terminal domain of AMH.

In one embodiment, the proAMH assay further comprises:
(i) contacting the biological sample with the first binding agent such that it binds to proAMH and AMH_{N,C};
(ii) contacting the bound proAMH and AMH_{N,C} with the splitting reagent in an amount sufficient to split the AMH_{N,C} complex into AMH_{N} and AMH_{C}; and
(iii) contacting the bound proAMH and AMH_{N} or AMH_{C} with the second binding agent and measuring the level of bound proAMH.

Importantly, the proAMH assay according to this aspect of the present invention uses the non-covalent association between the cleaved AMH species, namely the AMH_{N,C} complex, to distinguish between proAMH and AMH_{N,C}. To explain this concept further, and in reference to FIG. 2 which describes an ELISA assay format which is to be used for the purpose of illustration only and should not be considered limiting to the present invention, a first binding agent (in this case antibody) which binds to the N-terminal domain of AMH is bound to a solid support such as, for example, an ELISA plate well, and is used to capture proAMH and AMH_{N,C} present in a biological sample such as plasma or serum. Following standard wash step(s) to remove non-specifically bound analytes, a splitting reagent is then introduced to the assay to split the AMH_{N,C} complex into AMH_{N} and AMH_{C}. Since, in this example, the capture antibody only binds to the N-terminal domain of AMH, splitting of the AMH_{N,C} non-covalent complex via a splitting reagent causes dissociation of AMH_{C}, which is subsequently removed by further wash step(s). Finally, a second binding agent (in this case an antibody) is introduced to the assay which binds to the C-terminal domain of AMH. This detection antibody is specific for proAMH, since the AMH_{N,C} complex has been split and AMH_{C}, which previously formed part of the AMH_{N,C} complex, has been washed away and Is no longer present.

Antibodies, for example, polyclonal, monoclonal, bispecific, chimeric and humanized antibodies and antigen binding fragments are particularly preferred examples of binding agents according to the present invention.

In certain embodiments of the present invention, the first binding agent is bound to and immobilized on a solid support. Solid supports include, for example, multi-well plates, beads and tubes, and the binding agents according to the present invention may be conjugated directly to the solid support, for example, or by indirect binding. In an example of the latter case, antibodies or other polypeptides may be immobilized on particles or other solid supports, and that solid support immobilized to the device surface. This is described in more detail below under the heading “Immunoassay”.

A person skilled in the art will recognise that the detection antibody may comprise a detectable label. Accordingly, in other embodiments of the present invention, the second binding agent comprises a detectable label. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). The presence of the label may be quantified using standard techniques known to a person skilled in the art. The amount of detection antibody is directly proportional to the amount of proAMH present in the sample, thereby providing a quantative measure of the amount of proAMH in the sample.

A person skilled in the art will also recognise that the capture antibody in relation to the specific assay configuration depicted in Mg. 2 could be specific for the N-terminal or the C-terminal domain of AMH, provided that the detection antibody binds to the opposite domain of AMH. For the sake of clarity, and for avoidance of doubt, if the capture antibody binds to the N-terminal domain of AMH, then the detection antibody must bind to the C-terminal domain. Conversely, if the capture antibody binds to the C-terminal domain of AMH, then the detection antibody must bind to the N-terminal domain.

Accordingly, in certain embodiments of the present invention, the assay for proAMH comprises a first binding agent which binds to the C-terminal domain of AMH and a second binding agent which binds to N-terminal domain of AMH. In other embodiments, the first binding agent binds to the N-terminal domain of AMH and the second binding agent binds to the C-terminal domain of AMH.

The splitting reagent is used to split AMH_{N,C} into AMH_{N} and AMH_{C}. In other words, the splitting reagent must be capable of splitting non-covalent complexes. Examples of splitting reagents according to the present invention include, but are not limited to, salts, detergents, and chemical reagents sufficient to alter pH, such as acids or bases. Specific examples of splitting reagents include sodium deoxycholate, acetic acid and sodium dodecyl sulphate.

Importantly, the splitting reagent will not split proAMH into its constituent N- and C-terminal domains since these domains are covalently linked through a polypeptide bond. Accordingly, the bound proAMH will bind the detection antibody when added to the assay.

In the context of the examples provided above (i.e. ELISA assay format), the skilled person will appreciate that the splitting reagent must not:
(i) alter the conformation of proAMH such that it prevents proAMH from binding to capture and/or detection antibodies, or cause AMH to precipitate from solution;
(ii) alter the proAMH capture and/or detection antibodies in a manner that prevents these antibodies binding to proAMH; and
(iii) disrupt the assay format, for example, by stripping antibodies from the capture matrix, or by leaving residues that interfere with subsequent steps in the assay.

A person skilled in the art will further recognise that the AMH_{N,C} complex may be split before the sample is added to the assay. That way, in the ELISA assay format examples referred to above, the capture antibody will bind to proAMH, as well as AMH_{N} or AMH_{C}, depending on the capture antibody's specificity for AMH. By way of further illustration, if the capture antibody is specific for and binds to the N-terminal domain of AMH, then it will bind proAMH and AMH_{N}. Conversely, if the capture antibody is specific for and binds to the C-terminal domain of AMH, then it will bind proAMH and AMH_{C}. However, the skilled person will recognise that not all capture antibodies specific for the N- or C-terminal domains of AMH will bind to both proAMH and AMH_{N} or AMH_{C} (e.g. FIG. 6).

Accordingly, in another embodiment, the proAMH assay according to this aspect of the present invention further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH or AMH_{C} with the second binding agent and measuring the level of bound proAMH.

Irrespective of the assay's configuration, the specific measurement of proAMH is still achieved. The elimination of AMH_{N,C} from measurement by the assay using the splitting reagent is explained above. With respect to distinct AMH_{N} and AMH_{C} species (i.e. where not part of the AMH_{N,C} complex), the assay will not measure either species because AMH_{N} and AMH_{C} will exclusively bind to the capture antibody or the detection antibody, depending on the configuration of the assay, but importantly not both.

Further, to the extent that preAMH exists in the samples to be analysed, the assay and methods of the present invention will also detect preAMH as well.

A person skilled in the art will recognise that the binding agents and splitting reagents described herein may be used in related methods to determine the level of AMH in a biological sample, and in particular the level of proAMH and AMH_{N,C}.

Accordingly, in another aspect of the present invention there is provided a method for measuring the level of proAMH in a subject, the method comprising:
(i) obtaining a biological sample from the subject; and
(ii) measuring the level of proAMH in the sample using a first binding agent, a second binding agent and a splitting reagent.

In one embodiment of the method for measuring proAMH in the subject, the splitting reagent splits the AMH_{N,C} complex into AMH_{N} and AMH_{C}, and wherein when the first binding agent binds to the N-terminal domain of proAMH and AMH_{N,C}, the second binding agent is specific for and binds to the C-terminal domain of proAMH, or wherein when the first binding agent binds to the C-terminal domain of proAMH and AMH_{N,C}, the second binding agent is specific for and binds to the N-terminal domain of proAMH.

In yet a further aspect of the present invention there is provided assays and methods specific for the measurement of AMH_{N,C}. For example, and by way of Illustration only, one general concept underlying an AMH_{N,C} specific assay is also presented in FIG. 2, contrasted against conventional AMH ELISA assay platforms.

Briefly, the AMH_{N,C} assay according to this aspect of the present invention again uses the non-covalent association between the cleaved AMH species, namely the AMH_{N,C} complex, to distinguish between proAMH and AMH_{N,C}. In this example, a capture antibody which binds to the C-terminal domain of AMH is bound to a solid support such as, for example, an ELISA plate well, and is used to capture proAMH and AMH_{N,C} present in a biological sample such as plasma or serum. Following standard wash step(s) to remove non-specifically bound analytes, a splitting reagent is then introduced to the assay to split the AMH_{N,C} complex into AMH_{N} and AMH_{C}. Since, in this example, the capture antibody only binds to the C-terminal domain of AMH, the splitting reagent causes dissociation of AMH_{N}, which is subsequently removed by further wash step(s). Finally, a detection antibody is introduced to the assay which binds to an epitope contained within the C-terminal domain formerly of the AMH_{N,C} complex which epitope was previously shielded by AMH_{N}. In this example, the detection antibody will not bind to proAMH because the antibody is specific for AMH_{C}, thus providing a specific measure for AMH_{N,C}.

In one embodiment, the antibody specific for AMH_{C} is Goat anti-rat AMH/MIS antibody (R&D systems, Catalogue# AF1446). This is demonstrated in FIG. 6 where the antibody binds to rhAMH_{C} (lane 2), but not to rhAMH (proAMH; lanes 3 and 4) under non-reducing/non-denaturing/soluble conditions.

A person skilled in the art would recognise that the AMH_{N,C}, assay could alternatively be configured using a capture antibody specific for the N-terminal of AMH and a detection antibody specific for AMH_{N}, provided that the detection antibody does not also bind to proAMH. Alternatively, the skilled person would also recognise that a non-antibody detection means could be employed, for example, using a labelled form of AMH_{N} or AMH_{C}.

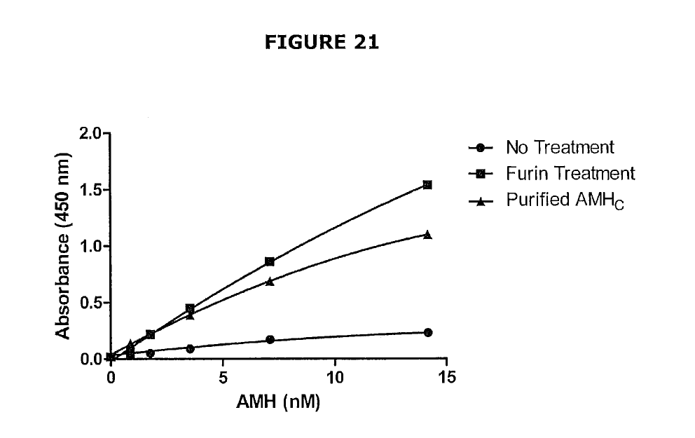

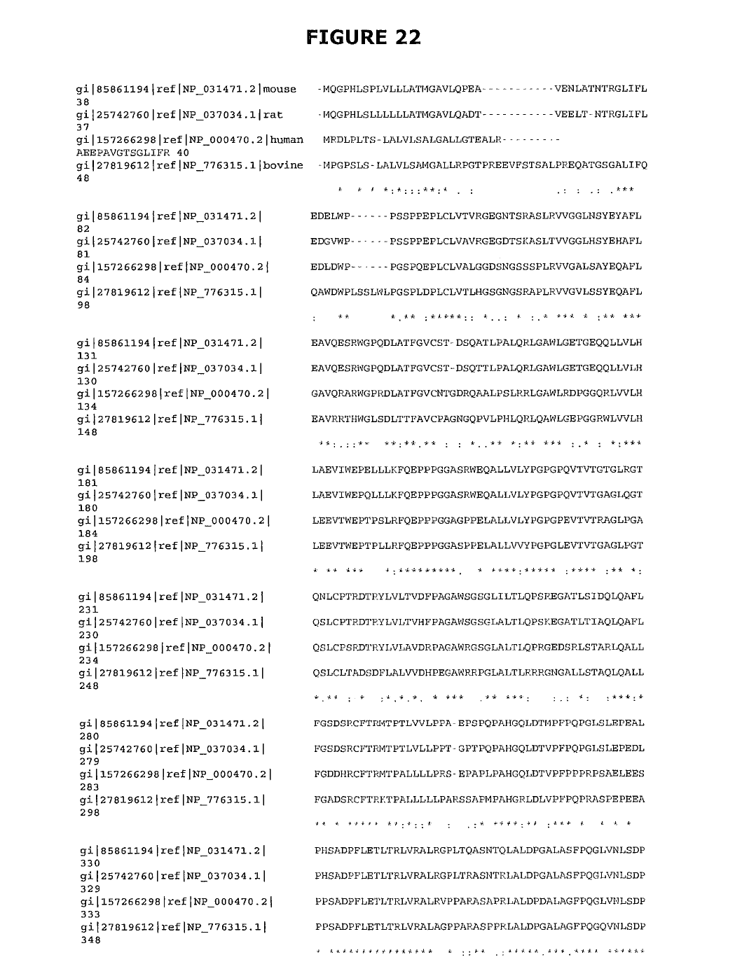

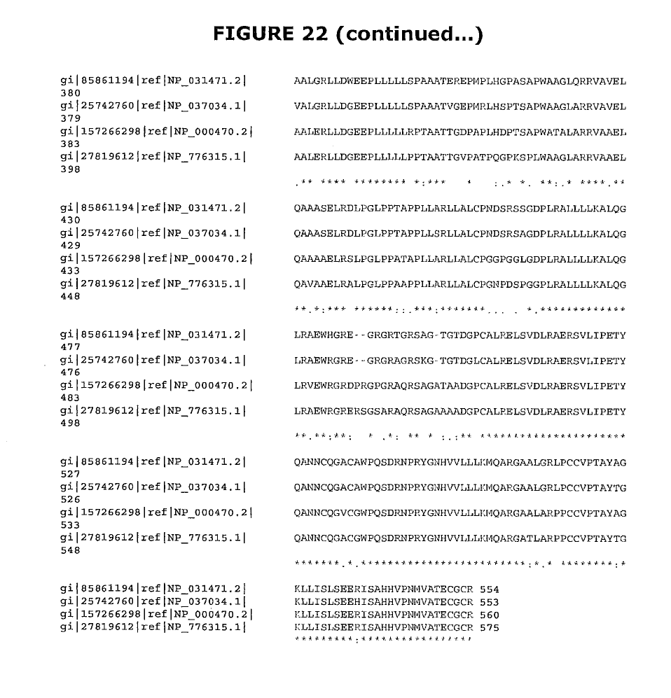

A person skilled in the art would also recognise that the sequence in which these assay steps are performed may be varied. For example, the biological sample (e.g. plasma or serum) may undergo pre-treatment with the splitting reagent, such as deoxycholate, prior to the capture and detection steps. This particular configuration or sequence of assay steps is demonstrated in Example 11/FIG. 21 which illustrates a working AMH_{N,C} specific ELISA assay format.

Accordingly, in a further aspect the present invention provides an assay for AMH_{N,C} In a biological sample comprising proAMH and AMH_{N,C} obtained from a subject, the assay comprising measuring the level of AMH_{N,C} using a first binding agent, a second binding agent and a splitting reagent, wherein the first binding agent binds to proAMH and AMH_{N,C}, the second binding agent binds to AMH_{N} or AMH_{C} but does not bind to proAMH or AMH_{N,C} and wherein the splitting reagent splits the AMH_{N,C} complex into AMH_{N} and AMH_{C}.

In one embodiment, the AMH_{N,C} assay further comprises:
(i) contacting the biological sample with the first binding agent such that it binds to proAMH and AMH_{N,C};
(ii) contacting the bound proAMH and AMH_{N,C} with the splitting reagent in an amount sufficient to spilt the AMH_{N,C} complex into AMH_{N} and AMH_{C}; and
(iii) contacting the bound proAMH and AMH_{N} or AMH_{C} with the second binding agent and measuring the level of bound AMH_{N} or AMH_{C},
   wherein the level of bound AMH_{N} or AMH_{C} corresponds to the amount of AMH_{N,C} in the sample.

In another embodiment, the AMH_{N,C} assay further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound AMH_{N} or AMH_{C},
   wherein the level of bound AMH_{N} or AMH_{C} corresponds to the amount of AMH_{N,C} in the sample.

In a related aspect of the present invention there is provided a method for measuring the level of AMH_{N,C} In a subject, the method comprising:
(i) obtaining a biological sample from the subject; and
(ii) measuring the level of AMH_{N,C} in the sample using a first binding agent, a second binding agent and a splitting reagent.

In one embodiment of the method for measuring AMH_{N,C} in the subject, the first binding agent binds to proAMH and AMH_{N,C}, the second binding agent binds to AMH_{N} or AMH_{C} but does not bind to proAMH or AMH_{N,C}, and wherein the splitting reagent is sufficient to split the AMH_{N,C} complex into AMH_{N} and AMH_{C}.

In another embodiment of the method for measuring AMH_{N,C} in the subject, the method further comprises:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(ii) contacting the bound proAMH and AMH_{N} or AMH_{C} with the second binding agent and measuring the level of bound AMH_{N} or AMH_{C},
   wherein the level of bound AMH_{N} or AMH_{C} corresponds to the amount of AMH_{N,C} in the sample.

In certain embodiments of the AMH_{N,C} assays and methods described herein, the first binding agent is immobilised on a solid substrate.

In other embodiments, the first binding agent is an antibody or antigen binding fragment thereof which binds to the N-terminal or C-terminal domain of AMH, provided that it binds to both proAMH and AMH_{N,C}.

The antibody or antigen binding fragment thereof may be selected from the group consisting of a polyclonal, monoclonal, bispecific, chimeric and humanized antibody or an antigen binding fragment thereof.

In certain other embodiments, the second binding agent comprises a detectable label. In one embodiment, the second binding agent is an antibody or antigen binding fragment that binds to AMH_{C} or AMH_{N}. Again, the antibody or antigen binding fragment thereof may be selected from the group consisting of a polyclonal, monoclonal, bispecific, chimeric and humanized antibody or an antigen binding fragment thereof. In one particular embodiment, the second binding agent is Goat anti-rat AMH/MIS antibody (R&D systems, Catalogue# AF1446).

In other embodiments, the second binding agent is a labelled form of AMH_{N} or AMH_{C}.

In addition to the proAMH assay configuration described earlier, the present invention also contemplates an assay for proAMH comprising a binding agent that is specific for proAMH and does not bind to any other form of AMH, including for example, AMH_{N,C}, AMH_{N} and AMH_{C}. That is, the binding agent exclusively binds proAMH.

Accordingly, in another aspect of the present invention there is provided an assay for proAMH in a biological sample comprising proAMH and AMH_{N,C} obtained from a subject, the assay comprising measuring the level of proAMH in the sample using a binding agent specific for proAMH.

In one embodiment, the assay according to this aspect of the present invention comprises:
(i) contacting the biological sample with the binding agent such that it binds to proAMH; and
(ii) measuring the level of bound proAMH in the sample.

In a related aspect of the present invention there is provided a method for measuring the level of proAMH in a subject, the method comprising:
(i) obtaining a biological sample from the subject;
(ii) measuring the level of proAMH in the sample using a binding agent specific for proAMH.

In certain embodiments of the assays and methods according to this aspect of the present invention, the binding agent is an antibody or an antigen binding fragment, such as, for example, a polyclonal, monoclonal, bispecific, chimeric and humanized antibody or antigen binding fragment thereof. A monoclonal antibody or antigen binding fragment thereof is particularly preferred.

In a further embodiment, the antibody or antigen binding fragment specific for proAMH binds to an epitope spanning the cleavage site between the N- and C-terminal domains of AMH (i.e. an epitope spanning residues Arg⁴⁵¹Ser⁴⁵² of the human propeptide of AMH).

The present invention also provides a quantitative measurement for AMH_{N,C} which is performed using a combination of a conventional assay for AMH and a specific assay for proAMH, as described herein. The Applicants demonstrate herein that the total AMH in a biological sample is predominantly comprised of proAMH and AMH_{N,C}, with no or only trace levels of AMH_{N} or AMH_{C} observed (FIGS. 5, 6, 7, 8). Accordingly, using conventional assays to measure the total amount of AMH in combination with an assay specific for proAMH provides the ability to measure AMH_{N,C}.

Accordingly, in yet a further aspect, the present invention provides an assay for AMH_{N,C} In a biological sample comprising proAMH and AMH_{N,C} obtained from a subject, the assay comprising:
(i) measuring the amount of total AMH in a sample; and
(ii) measuring the amount of proAMH in the sample
   wherein the amount of AMH_{N,C} in the sample represents the difference between total AMH and proAMH measured in the sample.

In a related aspect, the present invention also provides a method for measuring the amount of AMH_{N,C} in a subject, the method comprising:
(i) obtaining a biological sample from the subject;
(ii) measuring the amount of total AMH in the sample; and
(iii) measuring the amount of proAMH in the sample

wherein the amount of AMH_{N,C} in the sample represents the difference between total AMH and proAMH measured in the sample.

In certain embodiments, the amount of total AMH in the sample is determined using a conventional assay for AMH, including for example, the Beckman Coulter (Catalogue #A79765) and the ANSH Labs AMH ELISA (Catalogue #A79765).

In other embodiments, the amount of proAMH in the sample is determined using an assay described herein.

In yet another aspect, the present invention provides an assay for measuring total AMH in a biological sample comprising proAMH and AMH_{N,C}, the assay comprising:
(i) measuring the amount of proAMH in the sample; and
(ii) measuring the amount of AMH_{N,C} In the sample
   wherein the total amount of AMH in the sample represents the sum of the amounts of proAMH and AMH_{N,C} measured in the sample.

In a related aspect in a related aspect, the present invention also provides a method for measuring total AMH in a biological sample comprising proAMH and AMH_{N,C}, the method comprising:
(i) obtaining a biological sample from the subject;
(i) measuring the amount of proAMH in the sample; and
(iii) measuring the amount of AMH_{N,C} in the sample
   wherein the total amount of AMH in the sample represents the sum of the amounts of proAMH and AMH_{N,C} measured in the sample.

In certain embodiments, the amount of proAMH in the sample is determined using an assay or method described herein.

In other embodiments, the amount of AMH_{N,C} in the sample is determined using an assay or method described herein.

The present invention also contemplates dual purpose or multi-site assay formats for the detection and measurement of different forms of AMH, including proAMH and AMH_{N,C}. The configuration of multi-site assays, including multi-site ELISA formats, for the detection and measurement of different analytes would be well known to a person skilled in the art. In this specification, the terms dual purpose, multi-site and multiplex assays may be used interchangeably.

Accordingly, in yet another aspect of the present invention there is provided a dual purpose assay or method for detection and measurement of different species of AMH in a biological sample obtained from a subject, the dual purpose assay or method comprising any combination of the assays or methods described herein. In one embodiment, the dual purpose assay or method detects and measures both proAMH and AMH_{N,C}. In other embodiments, detection and measurement of the different species of AMH may be achieved simultaneously, that is a signal output for each species of AMH to be detected and measured is generated by the same assay at the same time.

In yet another aspect the present invention provides kits comprising a dual purpose assay or method described herein.

### Immunoassays

In general, immunoassays involve contacting a sample containing or suspected of containing a peptide biomarker of Interest with at least one antibody that specifically binds to the biomarker. A signal is then generated indicative of the presence or amount of complexes formed by the binding of peptides in the sample to the antibody. The signal is then related to the presence or amount of the peptide biomarker in the sample (quantitatively, semi-quantitatively or qualitatively). Numerous methods and devices are well known to the skilled artisan for the detection and analysis of peptide biomarkers. For example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods according to the present invention may utilize labelled molecules in various sandwich, competitive, or non-competitive assay formats to generate a signal that is related to the presence or amount of AMH in a sample, including, for example, proAMH and AMH_{N,C}. Suitable assay formats used for the present invention include in particular, enzyme-linked immunoassays (ELISA), radioimmunoassays (RIAs), competitive binding assays, and the like. Also contemplated are chromatographic, mass spectrographic, and protein “blotting” methods. Additionally, certain methods and devices, such as biosensors and optical immunoassays, may be employed to determine the presence or amount of analytes without the need for a labelled molecule. For example, refer to U.S. Pat. Nos. 5,631,171 and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the Immunoassay analyzers that are capable of performing immunoassays described here, as an example of the present invention.

Antibodies or other polypeptides may be immobilized onto a variety of solid supports for use in the assays and methods of the present invention. Solid supports or phases that may be used to immobilize specific binding agents include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colour spot. Antibodies or other polypeptides may be bound to specific zones of assay devices either by conjugating directly to an assay device surface, for example, or by indirect binding. In an example of the latter case, antibodies or other polypeptides may be immobilized on particles or other solid supports, and that solid support immobilized to the device surface.

Biological assays require methods for detection, and one of the most common methods for quantitation of results is to conjugate a detectable label to a protein that has affinity for one of the components in the biological system or sample being studied. In the assays and methods of the present invention, the detectable label is typically conjugated to a binding agent, such as an antibody. Binding of AMH to an antibody to form a complex can be detected directly or indirectly. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

By way of illustration, horseradish peroxidase for example can be incubated with substrates such as o-Phenylenediamine Dihyhydrochloride (OPD) and peroxide to generate a coloured product whose absorbance can be measured, or with luminol and peroxide to give chemiluminescent light which can be measured in a luminometer as is known in the art. Biotin or digoxin can be reacted with binding agents that bind strongly to them. For example, the proteins avidin and streptavidin will bind strongly to biotin. A further measurable label is then covalently bound or linked thereto either by direct reaction with the protein, or through the use of commonly available crosslinking agents such as carbodilmide, or by addition of chelating agents.

Detection also includes fluorescence resonance energy transfer (FRET) between fluorescent labels, particularly in dual assay formats according to the present invention for the simultaneous measurement of, for example, proAMH and AMH_{N,C}.

As such, the present invention also contemplates the analysis of different species of AMH, such as the detection and measurement of proAMH and AMH_{N,C} for example, using multi-site assay formats, as will be known to a person skilled in the art (see for example U.S. Pat. No. 7,541,160).

Generation of a signal from the label can be performed using various optical, acoustical, and electrochemical methods well known in the art. As described herein, examples of detection modes include fluorescence, radiochemical detection, reflectance, absorbance, amperometry, conductance, impedance, interferometry, ellipsometry, etc. This list is not meant to be limiting. Antibody-based biosensors may also be employed to determine the presence or amount of analytes that optionally eliminate the need for a labelled molecule.

Immunoassay analysers are also well known and Include Beckman Access, Abbott AxSym, Roche ElecSys and Dade Behring Status systems amongst others that are well described.

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Malelmides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

Sandwich type assays (a type of competitive binding assay) have greater specificity, speed and greater measuring range. In this type of assay an excess of the primary antibody to AMH, such as proAMH or AMH_{N,C}, is attached to the well of an ELISA plate, bead or tube via adsorption, covalent coupling, or a second antibody, as described above for solid phase competition binding assays. Sample fluid or extract is contacted with the antibody attached to the solid phase. Because the antibody is in excess this binding reaction is usually rapid. A detection antibody to AMH is also incubated with the sample either simultaneously or sequentially with the primary antibody. In certain embodiments, the detection assay is not added to the assay until the AMH_{N,C} complex has been split. The detection antibody is chosen to bind to a site on AMH that is different from the binding site of the primary antibody. These two antibody reactions result in a sandwich with the AMH from the sample sandwiched between the two antibodies. The detection antibody is usually labelled with a readily measurable compound as detailed above. Alternatively a labelled third antibody that binds specifically to the detection antibody may be contacted with the sample. After washing away the unbound material the bound labelled antibody can be measured and quantified by methods outlined for competitive binding assays.

In certain embodiments of the present invention, various types of immunoassays are used, which may include a competitive type of immunoassay. Examples of competitive immunoassays include an enzyme immunoassay or enzyme-linked immunosorbent assay (EIA or ELISA), a fluorescent immunoassay, a radiometric or radioimmunoassay (RIA), a magnetic separation assay (MSA), a lateral flow assay, a diffusion immunoassay, an immunoprecipitation assay, an immunosorbent or “antigen-down” assay using an analyte bound to a solid support, or an agglutination assay. In one such assay, a sample contains an unknown amount of analyte to be measured, which may be a protein such as proAMH or AMH_{N,C}, or both. The analyte may also be termed an antigen. The sample may be spiked with a known or fixed amount of labelled analyte. The spiked sample is then incubated with an antibody that binds to the analyte, such as proAMH or AMH_{N,C}, so that the analyte in the sample and the labelled analyte added to the sample compete for binding to the available antibody binding sites. More or less of the labelled analyte will be able to bind to the antibody binding sites, depending on the relative concentration of the unlabelled analyte present in the sample. Accordingly, when the amount of labelled analyte bound to the antibody is measured, it is inversely proportional to the amount of unlabelled analyte in the sample. The amount of analyte in the original sample may then be calculated based on the amount of labelled analyte measured, using standard techniques known in the art.

In another type of competitive immunoassay, an antibody that binds to the analyte, such as proAMH or AMH_{N,C} may be coupled with or conjugated to a ligand, wherein the ligand binds to an additional antibody added to the sample. One example of such a ligand includes fluorescein. The additional antibody may be bound to a solid support. The additional antibody binds to the ligand coupled with the antibody that binds in turn to the analyte or alternatively to the labelled analyte, forming a mass complex which allows isolation and measurement of the signal generated by the label coupled with the labelled analyte.

In another type of competitive immunoassay, the analyte to be measured, such as proAMH or AMH_{N,C}, may be bound to a solid support, and Incubated with both an antibody that binds to the analyte and a sample containing the analyte to be measured. The antibody binds to either the analyte bound to the solid support or to the analyte in the sample, in relative proportions depending on the concentration of the analyte in the sample. The antibody that binds to the analyte bound to the solid support is then bound to another antibody, such as anti-mouse IgG, that is coupled with a label. The amount of signal generated from the label is then detected to measure the amount of antibody that bound to the analyte bound to the solid support. Such a measurement will be inversely proportional to the amount of analyte present in the sample. Such an assay may be used in a microtiter plate format.

An additional embodiment of the present invention discloses a method for measuring an amount of AMH, including proAMH or AMH_{N,C}, or both, in a sample.

Embodiments of the Invention as disclosed herein may be used to perform immunoassays referred to as immunometric, “two-site” or “sandwich” immunoassays, wherein the analyte may be bound to or sandwiched between two antibodies that bind to different epitopes on the analyte, such as proAMH or AMH_{N,C}. Representative examples of such immunoassays include enzyme immunoassays or enzyme-linked immunosorbent assays (EIA or ELISA), immunoradiometric assays (IRMA), fluorescent immunoassays, lateral flow assays, diffusion immunoassays, immunoprecipitation assays, and magnetic separation assays (MSA). In one such assay, a first antibody, which may be described as the “capture” antibody, may be bound to a solid support, for which examples have been listed above. The capture antibody may be bound to or coated on a solid support using procedures known in the art. Alternatively, the capture antibody may be coupled with a ligand that is recognized by an additional antibody that is bound to or coated on a solid support. Binding of the capture antibody to the additional antibody via the ligand then indirectly immobilizes the capture antibody on the solid support. An example of such a ligand is fluorescein. The second antibody, which may be described as the “detection” antibody, may be coupled with a label, which may comprise a chemiluminescent agent, a calorimetric agent, an energy transfer agent, an enzyme, a fluorescent agent or a radioisotope. The detection antibody may be coupled with or conjugated with a label using procedures known in the art. The label may comprise a first protein such as biotin coupled with the second antibody, and a second protein such as streptavidin that is coupled an enzyme. The second protein binds to the first protein. The enzyme produces a detectable signal when provided with substrate(s), so that the amount of signal measured corresponds to the amount of second antibody that is bound to the analyte. Horseradish peroxidase is an example of such an enzyme; possible substrates include TMB (3,3′, 5,5′-tetramethyl benzidine, OPD (o-phenylene diamine), and ABTS (2,2′-azino-bis(3-ethylbenzothiazoline-6-sulfonic acid).

Sandwich immunoassays or sandwich ELISAs are particularly suited for use in the present invention.

A dipstick type assay may also be used. These assays are well known in the art. They may for example, employ small particles such as gold or coloured latex particles with specific antibodies attached. The liquid sample to be measured may be added to one end of a membrane or paper strip preloaded with the particles and allowed to migrate along the strip. Binding of the antigen (such as proAMH and/or AMH_{N,C}) in the sample to the particles modifies the ability of the particles to bind to trapping sites, which contain binding agents for the particles such as antigens or antibodies, further along the strip. Accumulation of the coloured particles at these sites results in colour development that are dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and Incubation with substrates to produce colour, fluorescent or chemiluminescent light output will enable quantitation of antigen in the sample.

A radioimmunoassay (RIA) may also be used. In one RIA a radiolabelled antigen and unlabelled antigen are employed in competitive binding with an antibody. Common radiolabels include ¹²⁵I, ¹³¹I, ³H and ¹⁴C. Radioimmunoassays involving precipitation of AMH with a specific antibody and radiolabelled antibody binding protein can measure the amount of labelled antibody in the precipitate as proportional to the amount of the AMH in the sample. Alternatively, a labelled AMH species is produced and an unlabelled antibody binding protein is used. A biological sample to be tested is then added. The decrease in counts from the labelled AMH is proportional to the amount of AMH in the sample.

In RIA it is also feasible to separate bound AMH from free AMH. This may involve precipitating the AMH/antibody complex with a second antibody. For example, if the AMH/antibody complex contains rabbit antibody then donkey anti-rabbit antibody can be used to precipitate the complex and the amount of label counted. For example in an LKB, Gammamaster counter. See Hunt et al. Clin. Endocrinol. 1997 47:287-296.

### Antibodies and Antigen Binding Fragments

As noted above, antibody or antibodies as used herein refers to a peptide or polypeptide derived from, modelled after or substantially encoded by an Immunoglobulin gene or immunoglobulin genes, or fragments thereof, capable of specifically binding an antigen or epitope. Refer, for example, to Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. As foreshadowed in the definition section of this specification, the term antibody includes antigen binding fragments such as, for example, fragments, subsequences, complementarity determining regions (CDRs) that retain capacity to bind to an antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an Isolated complementarity determining region (CDR). Single chain antibodies are also included by reference in the term “antibody.”

For a further discussion of antibodies and fragments see for example Proc. Natl. Acad. Sci. USA 81: 6851-6855 (1984), Protein Eng 8(10) 1057-1062 (1995); The Pharmacology of Monoclonal Antibodies, vol. 113, Springer-verlag 1994, Rosenburg and Moore Eds; Proc. Natl. Acad. Sci USA 90: 6444-6448 (1993); Nature 321: 522-525 (1986); Nature 332: 323-329 (1988), and WO 2005/003154, all of which are incorporated herein in their entirety.

Also included is antiserum obtained by immunizing an animal such as a mouse, rat or rabbit with an AMH, such as for example, proAMH, AMH_{N} or AMH_{C}, or fragments or antigenic variants thereof. In brief, methods of preparing polyclonal antibodies are known to the skilled artisan. Polyclonal antibodies can be raised in a mammal, for example, by one or more injections of an Immunizing agent and, if desired, an adjuvant. Typically, the immunizing agent and/or adjuvant will be injected in the mammal by multiple subcutaneous or Intraperitoneal injections. The immunizing agent may include proAMH, AMH_{N} or AMH_{C}, fragments, antigenic variants thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants that may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art. For example, refer to Kohler and Milstein (1975) Nature (5517) 256, 495-497), U.S. Pat. No. 4,196,265, U.S. Pat. No. 4,816,567. The hybridoma cells may be cultured in a suitable culture medium, alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines that secrete the antibody of Interest. Sequences of proAMH, AMH_{N} or AMH_{C} or fragments or antigenic variants thereof may be used in screening.

Well known means for establishing binding specificity of monoclonal antibodies produced by the hybridoma cells include immunopredpltation, radiolinked immunoassay (RIA), enzyme-linked immunoabsorbent assay (ELISA) and Western blot. (Lutz et al. (1988) Exp. Cell. Res. 175:109-124). For example, as noted above, the binding affinity of the monoclonal antibody can, for example, be determined by the Scatchard analysis described in Munson et al. (1980) Anal Biochem 107: 220. Samples from immunised animals may similarly be screened for the presence of polyclonal antibodies.

Monodonal antibodies can also be obtained from recombinant host cells. DNA encoding the antibody can be obtained from a hybridoma cell line. The DNA is then placed into an expression vector, transfected into host cells (e.g., COS cells, CHO cells, E. coli cells) and the antibody produced in the host cells. The antibody may then be isolated and/or purified using standard techniques.

The monoclonal antibodies or fragments may also be produced by recombinant DNA means (see for example U.S. Pat. No. 4,816,567). DNA modifications such as substituting the coding sequence for human heavy and light chain constant domains in place of the homologous murine sequences (U.S. Pat. No. 4,816,567 above) are also possible. The antibodies may be monovalent antibodies. Methods for preparing monovalent antibodies are well known in the art (see, for example, U.S. Pat. Nos. 5,334,708, 5,821,047, and 7,476,724). Production of chimeric (U.S. Pat. No. 4,816,567), bivalent antibodies (U.S. Pat. No. 5,843,708) and multivalent antibodies are also contemplated herein (U.S. Pat. No. 6,020,153).

Other known art techniques for monoclonal antibody production such as from phage libraries, may also be used. See for example, Nature 352: 624-628 (1991).

The monoclonal antibodies secreted by the cells may be isolated or purified from the culture medium or ascites fluid by conventional immunoglobulin purification procedures such as, for example, reverse phase HPLC, protein A-Sepharose, hydroxyapatite chromatography, gel electrophoresis, dialysis, or affinity chromatography. See, for example, Scopes, Protein Purification: Principles and Practice, Springer-Verlag, NY (1982).

Bispecific antibodies may also be useful. These antibodies are monoclonal, preferably human or humanized, antibodies that have binding specificities for at least two different antigens. Antibodies with greater than two specificities for example trispecific antibodies are also contemplated herein.

Antibodies used in the Immunoassays described herein preferably specifically bind to AMH, such as proAMH or AMH_{N,C}. The term “specifically binds” is not intended to indicate that an antibody binds exclusively to its intended target since, as noted above, an antibody binds to any polypeptide displaying the epitope(s) to which the antibody binds. Rather, an antibody “specifically binds” if Its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In other embodiments, antibodies bind with affinities of at least about ⁻⁶, or 10⁻⁷M, or at least about 10⁻⁸M, or 10⁻⁹M, or 10⁻¹⁰, or 10⁻¹¹ or 10⁻¹²M.

Affinity is calculated as K_{d}=k_{off}/kₒₙ (k_{off} is the dissociation rate constant, Kₒₙ s the association rate constant and K_{d} is the equilibrium constant). Affinity can be determined at equilibrium by measuring the fraction bound (r) of labelled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art (van Erp et al. (1991) J. Immunoassay 12: 425-43; Nelson and Griswold (1988) Comput. Methods Programs Biomed. 27: 65-8).

Numerous publications discuss the use of phage display technology to produce and screen libraries of polypeptides for binding to a selected analyte (Cwirla et al. (1990) Proc. Natl. Acad. Sci. USA 87, 6378-82; Devlin et al. (1990) Science 249, 404-6; Scott and Smith (1990) Science 249, 386-88; and U.S. Pat. No. 5,571,698). A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target binds to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. For example, refer to U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labelled secondary antibody (for example, an anti-mouse antibody conjugated to alkaline phosphatase if the raised antibodies are mouse antibodies) is added to the wells and incubated for about 30 min and then washed. Substrate is added to the wells and a colour reaction will appear where antibody to the immobilized polypeptide(s) is present.

The antibodies so identified may then be further analysed for affinity and specificity In the assay design selected. In the development of immunoassays for a target protein, the purified target protein acts as a standard with which to judge the sensitivity and specificity of the Immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

### Kits

In various related aspects, the present invention also relates to devices and kits for performing the assays and methods described herein. Suitable kits comprise reagents sufficient for performing an assay for at least one of the described AMH species, together with instructions for performing the described threshold comparisons. For example, kits will be formatted for assays known in the art, and in particular, ELISA assays.

In one aspect of the present invention there is provided a kit for measuring proAMH in a biological sample obtained from a subject comprising proAMH and AMH_{N,C}, the kit comprising a first binding agent, a second binding agent, a splitting reagent and instructions for how to measure the level of proAMH in the sample.

According to this aspect of the present invention, the components of the kit are further characterised:
(i) the splitting reagent is capable of splitting the AMH_{N,C} complex into AMH_{N} and AMH_{C}; and
(ii) when the first binding agent binds to the N-terminal domain of proAMH and AMH_{N,C}, the second binding agent is specific for and binds to the C-terminal domain of proAMH; or
(iii) when the first binding agent binds to the C-terminal domain of proAMH and AMH_{N,C}, the second binding agent is specific for and binds to the N-terminal domain of proAMH.

In another aspect of the present invention there is provided a kit for measuring AMH_{N,C} In a biological sample comprising proAMH and AMH_{N,C} obtained from a subject, the kit comprising a first binding agent, a second binding agent, a splitting reagent, and instructions for how to measure the level of AMH_{N,C} in the sample.

According to this aspect of the present invention, the components of the kit are further characterised:
(i) the splitting reagent splits the AMH_{N,C} complex into AMH_{N} and AMH_{C}; and
(ii) when the first binding agent binds to proAMH and AMH_{N,C}, the second binding agent is specific for and binds to AMH_{N} or AMH_{C}.

In certain embodiments, reagents for performing such assays are provided in an assay device, and such assay devices may be included in such a kit. For example, preferred reagents can comprise one or more solid phase antibodies, the solid phase antibody comprising an antibody that detects the AMH species bound to a solid support.

Accordingly, in certain embodiments of the present invention, the first binding agent is immobilised on a solid support.

In the case of sandwich immunoassays, such reagents can also include one or more detectably labelled antibodies, the detectably labelled antibody comprising an antibody that detects the Intended AMH species bound to a detectable label. Additional optional elements that may be provided as part of an assay device are described hereinafter. Detectable labels may include molecules that are themselves detectable (e.g., fluorescent moieties, electrochemical labels, electrochemical luminescence (ecl) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labelled antibody that binds to the second antibody, biotin, digoxigenin, maltose, oligohistidine, 2,4-dintrobenzene, phenylarsenate, ssDNA, dsDNA, etc.).

As such, in other embodiments of the present invention, the second binding agent comprises a detectable label.

In yet another aspect of the present invention there is provided a kit for measuring the level of proAMH in a biological sample comprising proAMH and AMH_{N,C} obtained from a subject, the kit comprising a binding agent specific for proAMH.

In one embodiment, the binding agent specific for proAMH binds to an epitope that spans the cleavage site between the N- and C-terminal domains of proAMH.

As described herein, the binding agents comprised within the kits of the present invention may include an antibody or an antigen binding fragment thereof, for example, a monoclonal antibody or antigen binding fragment thereof. A detailed description with respect to binding members, including antibodies and antigen binding fragments is described elsewhere herein,

With respect to the kit for measuring the level or amount of AMH_{N,C} in a biological sample, the second binding agent may be a labelled form of AMH_{N} or AMH_{C}.

In a further embodiment, the splitting reagent of the kits described herein comprises deoxycholate.

In certain aspects, the present invention provides kits for the analysis of AMH, including proAMH and AMH_{N,C}. The kit comprises reagents for the analysis of at least one test sample. The kit can also include devices and Instructions for performing one or more of the diagnostic and/or prognostic correlations described herein. Preferred kits will comprise an antibody pair for performing a sandwich assay, or a labelled species for performing a competitive assay, for an analyte, such as proAMH or AMH_{N,C}. Preferably, an antibody pair comprises a first antibody conjugated to a solid phase and a second antibody conjugated to a detectable label, wherein each of the first and second antibodies will bind different forms of AMH, such as proAMH or AMH_{N,C}. Typically, and for the sake of specificity, each of the antibodies used In the kits of the present invention include monoclonal antibodies. The instructions for use of the kit and performing the correlations can be In the form of labelling, which refers to any written or recorded material that is attached to, or otherwise accompanies a kit at any time during its manufacture, transport, sale or use. For example, the term labelling encompasses advertising leaflets and brochures, packaging materials, instructions, audio or video cassettes, computer discs, as well as writing imprinted directly on kits.

Further encompassed within the scope of the present invention is kits comprising dual purpose or multi-site assays for the detection and measurement of different species of AMH, including proAMH and AMH_{N,C}. That is, the present invention provides assays and kits capable of simultaneously determining the presence and amount of different species of AMH, such as AMH_{N,C} and proAMH, in a biological sample which has been obtained from a subject. In certain embodiments, the present invention provides dual purpose assays and kits comprising dual purpose assays for the simultaneous measurement of proAMH and AMH_{N,C}, wherein the assay comprises any combination of the assays described herein.

### Relative Abundance of AMH Species in the Blood of Healthy Individuals

The relative abundance of proAMH and AMH_{N,C} In the blood of healthy individuals recruited from the community was analysed using the assays, methods and kits described herein. The study incorporated a comparison between the sexes and between the major stages of the life cycle for each sex.

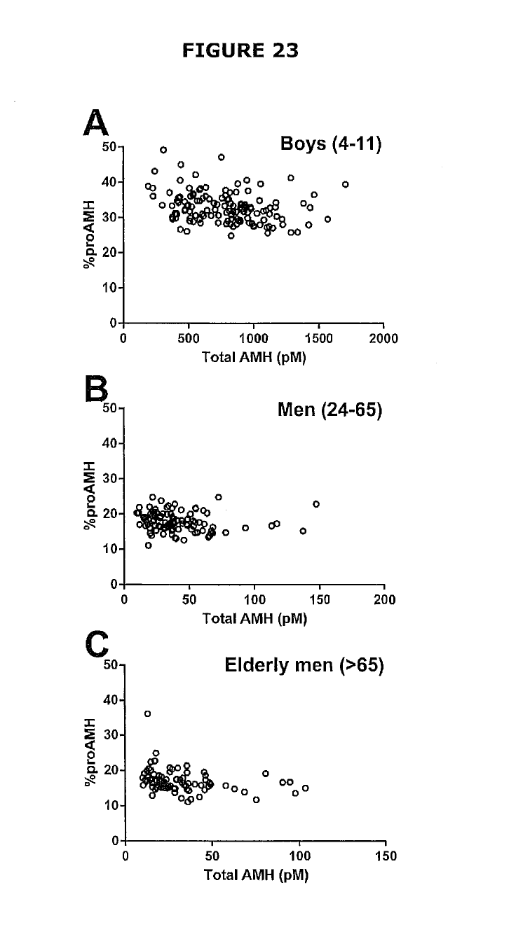

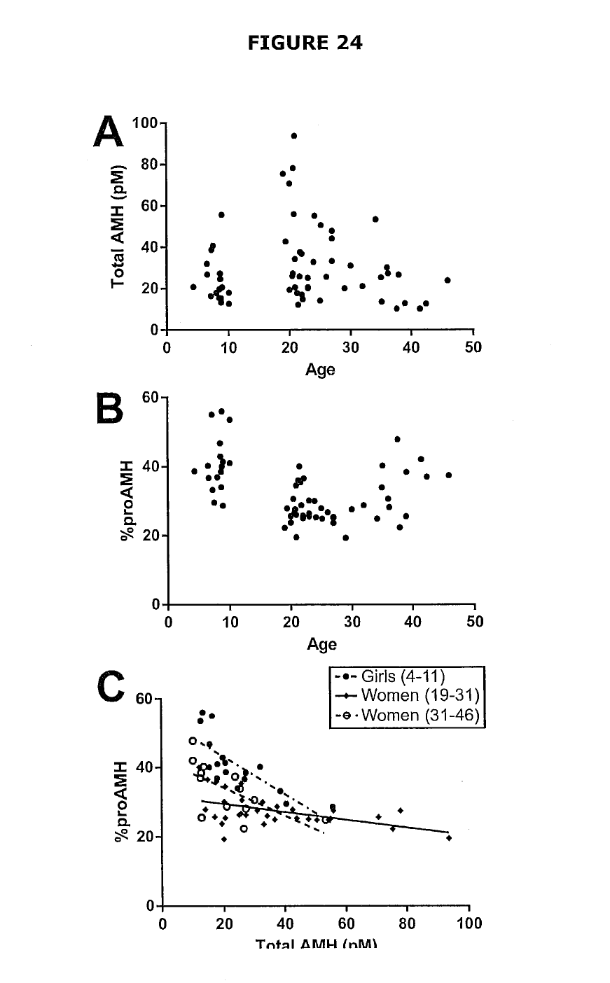

The results are presented in Example 12 (including Tables 3-5) and in FIGS. 23 and 24. These data reveal a number of interesting observations, namely that (i) proAMH levels can be measured in human serum from healthy individuals recruited from the community, (ii) the levels of proAMH, total AMH and % proAMH vary between individuals, and between distinct population groups; and (iii) the proAMH ELISA described herein generates data that is distinct from the existing commercially available AMH ELISAs.

The assays, methods and kits described herein are useful in the measurement of different forms of AMH present in a biological sample, for example serum. This information can be used to differentiate the biological functions of the various AMH species.

The Invention is further described with reference to the following Examples. It will be appreciated that the invention as claimed is not intended to be limited in any way by these examples.

### EXAMPLES

### Example 1

### Methods and Materials

### Study Participants

Study participants were healthy individuals recruited from the community in Dunedin, New Zealand in the categories of boys, men and women of reproductive age. A single post-menopausal woman was recruited as a negative control; post-menopausal women lack serum AMH (de Vet et al. (2002) Fertil Steril 77:357-362). Blood samples from plasma were collected in EDTA coated tubes (BD) and the plasma obtained by centrifugation at 2 000×g for 5 minutes. Serum was obtained by collection of blood into serum collection vacutainers (BD). The blood sat at room temperature for between 30 minutes and 1 hour, after which the serum was removed by centrifugation at 2 000×g for 5 minutes. Serum and plasma samples were divided into aliquots, snap frozen and stored at −80° C. until assay. The sex, age and blood AMH concentration of each participant is recorded in Table 1.

### Recombinant AMH

Recombinant human AMH (rhAMH) was produced from HEK293 cells transfected with a proAMH vector (Px'Therapeutics SA (Grenoble, France), under contract to the University of Otago). The resultant rhAMH was a mixture of proAMH, with lesser amounts of AMH_{N,C} (FIG. 3). Recombinant human AMH_{N} (rhAMH_{N}) was produced from HEK293 cells transfected with a AMH_{N} vector (Px'Therapeutics SA (Grenoble, France), under contract to the University of Otago). A solution of enriched In rhAMHN,C and diminished in proAMH was manufactured by cleaving batches of the rhAMH (PX'Therapeutics) with furin. The following conditions produce extensive cleavage, producing a solution with only trace levels of proAMH. Approximately, 10 nM rhAMH was reacted with 8 units of furin (New England Biolabs) in 40 μL of reaction buffer consisting of 100 mM HEPES, pH 7.4, 1.8 mM Ca²⁺, 140 mM Na⁺, 4 mM K⁺, 0.05% v/v Tween-20 for 24 hours at 37° C. The reaction was stopped by the addition of Lammli buffer and heating to 95° C. for 5 minutes. A second aliquot of rhAMH was subjected to the same treatment except that no furin was added to the reaction buffer. This used as a control for the furin treated material. rhAMH_{C} was purchased from R&D systems (1737-MS).

### Antibodies and Related Reagents

The anti-N terminal antibody was purchased from R&D systems (Catalogue#AF2748, anti-human AMH propeptide antibody). Epitopes from the C-terminus to the cleavage site of AMH were detected or precipitated with a either Goat anti-human AMH/MIS antibody (R&D systems, Catalogue# AF1737) (FIG. 5) and Goat anti-rat AMH/MIS antibody (R&D systems, Catalogue# AF1446) (FIG. 6). IRDye680 Donkey anti-Goat IgG antibody (Ucor) was used as the secondary antibody for western blot. Trueblot rabbit anti-goat Ig IP agarose beads (eBioscience) were used to precipitate the capture antibodies in the immunoprecipitation events. The wells, washing solutions and antibodies from the AMH gen II ELISA kit (Beckman Coulter) were used as reagents. Control IgG from non-immunised goats was obtained from Jackson Immuno Research Laboratories.

### Western Blotting

SDS PAGE was run on 10% tris-glycine bis-acrylamide gels with 4% stacking gel at 100 volts for 1.5 hours using the Xcell Surelock Mini-Cell system (Invitrogen). Proteins were transferred to 0.4 μm nitrocellulose membrane (Whatman GmbH) at 30 volts for 1 hour on ice. Blotting membranes were blocked with Odyssey blocking reagent (Licor) for 30 minutes. Primary antibodies were diluted to 0.1 μg/mL and Incubated overnight at 4° C. in blocking reagent. Blots were rinsed in Tris-buffered saline with 0.05% v/v Tween 20. After rinsing, IRDye680 Donkey anti-Goat IgG antibody (Licor) was applied at 0.66-1 μg/mL for 10-60 minutes before final rinsing and drying of the blotting membrane (FIGS. 4, 5, 7-10). The blotting membrane was imaged using the Odyssey fluorescent infrared scanner (Licor). The voltage (gain) to the photomultiplier tube was used to vary the intensity of the blot depending on the signal strength of the relevant bands. The grayscale has been inverted to present the Image as black-on-white images.

### Image Quantification

The density of the bands In some of the western blots were quantified without Image manipulation, using Image J software (NIH, http://rsbweb.nih.gov/ij/). The original image was then enhanced linearly to give visual confirmation of the densitometry measurements. This involved a linear stretch of the dynamic range of the image intensity such that the range of pixel intensities occupied by the histogram was linearly recalculated to occupy the full 256 bit-space with image J. The “dust and speckles” filter in Photoshop CS5 (Adobe) with a pixel radius of 6 and a threshold of 10 (Image resolution: 300 pixels/Inch) was used to reduce the auto-fluorescence of dust particles in the Image.

### Collection of Blood

Plasma: Blood samples were collected in EDTA coated tubes (BD) and the plasma obtained by centrifugation at 2 000×g for 5 minutes. Serum: 2 mL of blood was collected into BD vacutainers by either a registered phlebotomist or licensed medical practitioner. The blood sat room temperature for between 30 minutes and 1 hour, after which the serum was removed, divided into aliquots, snap frozen and stored at −80° C. Again, the sex, age and blood AMH concentration of each participant is recorded in Table 1.

Immunoprecipitation of AMH with Agarose Beads

AMH was immunoprecipitated with either polyclonal anti-human proAMH goat IgG (R&D systems, AF2748, amino acids 19-450) or a mixture of polyconal antibodies to the C-terminal peptide (amino acids 447-553): anti-human AMH/MIS goat IgG and goat anti-rat AMH/MIS antibody (R&D systems, AF1446, AF2748) (FIGS. 3-6). 1 mL of plasma was incubated with 1 μg of antibody at 37° C. overnight, together with the following preservatives: 10 μL of 3% sodium azide (Merck); 10 μL of 100 mM phenylmethanesulfonyl fluoride (PMSF, Sigma) and 40 μL of 25× complete mini protease inhibitor cocktail (Roche). 50 μL of rabbit anti-goat IgG Trueblot beads was then added to the solution, which was incubated for 1 hour at room temperature before the centrifugation at 6 700×g for 30 seconds. The supernatant was removed, and the beads washed 3 times in 0.01 M phosphate buffer (PB), and then heated to 95° C. in Laemmli buffer for 5 minutes. The released proteins were run on SDS-PAGE (10% bis-acrylamide with 4% stacking gel) for western blotting.

Enzymatic Deglycosylation of AMH from Human Blood

The primary antibody was cross-linked to the beads for the analysis of the gycosylation state of AMH to minimize the breadth of the IgG band (FIG. 8 FIGS. 9, 10). Rabbit anti-goat IgG Trueblot beads were incubated with goat anti-human AMH propeptide antibody for 1 hour at room temperature with agitation. The beads were rinsed twice with 0.01 M PB and twice with 0.2 M triethanolamine (BDH), pH 8.2. The associated proteins were cross-linked with 25 mM dimethyl pimelldate dlhydrochloride (Sigma), 0.2 M triethanolamine, pH 8.2 for 45 minutes at room temperature. The reaction was stopped with one quick rinse, followed by 1 hour of Incubation in 0.1 M ethanolamine (Sigma), pH 8.2. The beads were rinsed 2 times with 0.01 M PB and unbound antibody was eluted with 0.1 M glycine-HCl (BDH), pH 2.5 followed by 2 rinses in 0.01 M PB.

The plasma of boy B3 was diluted 1:2 with 0.01 M PB. The AMH was immunoprecipitated from the sample with 1 μg of goat anti-human AMH propeptide antibody cross-linked to rabbit anti-goat IgG Trueblot beads. Incubation was conducted overnight at 37° C. with 0.03% sodium azide (Merck), 1 μM phenylmenthanesulfonylfluoride (Sigma) and complete mini protease inhibitor cocktail (Roche) diluted as per manufacturer's instruction. The beads were then rinsed twice with 1 mL 0.01 M PB resuspended in 1 mL 0.01M PB and transferred to a new microtube. Enzymatic deglycosylation was conducted using the EDEGLY enzymatic deglycosylation kit (Sigma). To elute the precipitated AMH, the beads were pelleted, the supernatant was removed and 60 μL of ultra-pure water, 25 μL of the 5× reaction buffer and 2.2 μL of 5% SDS (Sigma) solution was added. The sample was heated to 95° C. for 5 minutes and the supernatant was transferred to new tubes in 21 μL aliquots. Disulfide bonds in the samples were reduced with 0.9 μL of 2-mercaptoethanol and heating to 95° C. for 5 minutes. To each sample, 1.2 μL of 15% triton X-100 and 0.5 μL of each required deglycosylation enzyme (β-N-acetylgicuosaminidase, Slalidase A, O-glycosldase, β(1→4)-galactosidase and/or PNGase F) were added followed by Incubation at 37° C. for varying lengths. Samples were analysed by western blot.

### Measurement of AMH Using Existing Commercial ELISA

The AMH concentration in the plasma and serum samples and the rhAMH were assayed using the Beckman Coulter AMH Generation II ELISA Kit (Beckman Coulter). Standard curves for interpolation were generated by fitting a quadratic curve to the absorbance values obtained for the AMH standard solutions (Beckman Coulter).

### Recovery of Proteins Captured by ELISA Wells

Serum or recombinant proteins were added to the ELISA wells, typically with a volume of 20 μL 100 μL of the manufacturer's assay buffer was immediately added. The wells were incubated for 1 hour at room temperature on an orbital shaker at 300 rpm. The wells were washed 10 times with 400 μL of ELISA wash buffer, using a ELISA plate washer. The wash buffer was removed and 40 μL of Laemmll buffer added to each well and covered with a pre-cut square of 96-well plate adhesive cover. The wells were incubated on a metal plate at 95° C. for 6 minutes, after which the wells were cooled on the bench. The plastic covers were removed from each well and placed on a 1.6 mL max recovery tube and pulse spun to transfer any condensate from the cover into the tube. Each well was then inverted into the recovery tube and spun. The tubes were then heated to 95° C. for 5 minutes and the samples analyzed by western blots.

### ProAMH ELISA

Samples containing AMH were applied to a Beckman Coulter (BC) AMH Gen II ELISA plate at a volume of 20 μL. Assay buffer was then added to the ELISA plate wells (100 μL) and the samples were incubated at room temperature for 1 hour on an orbital shaker at 300 rpm. The wells were washed 5 times with the ELISA kit wash buffer. Two changes of 150 μL of 0.2% sodium deoxycholate in 0.02 M Phosphate buffer pH 6.8, pre-warmed to 37° C. were applied to the wells and were incubated for 15 minutes each at 37° C. The deoxycholate is used to dissociate the AMH_{N,C} complex without affecting overall binding of the AMH species to the ELISA plate. The wells were washed 5 times with the ELISA kit wash buffer. 100 μL of AMH Gen II kit biotinylated detection antibody solution was applied then the plate was incubated at room temperature for 1 hour on an orbital shaker at 300 rpm. The wells were then washed 5 times with the ELISA kit wash buffer. Streptavidin-horse radish peroxidase conjugate was applied at 100 μL to the ELISA wells and was incubated at room temperature for 30 minutes on an orbital shaker at 300 rpm. The wells were washed 5 times with the ELISA kit wash buffer. TMB was applied at 100 μL to all wells and was incubated at room temperature for 8-12 minutes, after which 100 μL of stop-solution was applied. Absorbance was read at 450 nm and 570 nm for background correction. The 570 nm absorbance was subtracted from that at 450 nm to obtain the true absorbance reading.

### AMH_{N,C} ELISA

The AMH-Duoset capture antibody (R&D systems, catalogue #DY1737) was diluted to 4 μg/ml in 0.05 M Carbonate buffer pH 9.6-9.8 and 100 μL was applied to a 96-well plate 18 hours incubation at 4° C. The plate was washed 3 times with phosphate-buffered saline, pH 7.4 with 0.05% Tween 20 (Sigma, catalogue#P-1739) (PBS-T). The wells were Incubated for 30 minutes with 300 μL of phosphate-buffered saline, pH 7.4, 1% Tween 20 at room temperature. Concurrently, 10 μL of sample was mixed with 90 μL of 0.2% sodium deoxycholate in 0.02 M Phosphate buffer pH 6.8, pre-warmed to 37° C. and were incubated at 37° C. for 30 minutes. For recombinant preparations; ProAMH samples were non-furin treated, AMH_{N,C} samples were furin-treated and AMH_{C} samples were purified rh-AMH_{C} (R&D systems, catalogue #1737-MS). The plate was washed 3 times with PBS-T and 100 μL of deoxycholate-treated sample was added to the plate. The plate was incubated for 1 hour at room temperature and 30 minutes at 37° C. The plate was washed 3 times with PBS-T and 100 μL of AMH-Duoset detection antibody (R&D systems, catalogue #DY1737), diluted to 2 μg/mL in 1% BSA in PBS-T was applied to the wells and was incubated at room temperature for 1 hour. The plate was washed 3 times with PBS-T and 100 μL of Amersham Streptavidin-Biotinylated Horseradish Peroxidase Complex (GE healthcare, catalogue#RPN1051) diluted 1:2000 in PBS-T was applied for 15 minutes at room temperature. The plate was washed 3 times with PBS-T. TMB (KPL, catalogue#50-76-11) was applied at 100 μL to all wells and was incubated at room temperature for 15-20 minutes, after which 100 μL of 2M sulphuric acid was applied. Absorbance was read at 450 nm and 570 nm for background correction. The 570 nm absorbance was subtracted from that at 450 nm to obtain the true absorbance reading.

### Example 2

### Validation of AMH Antibodies for Western Blotting

The molecular weights and Immunoreactivity of proAMH, AMH_{N}, AMH_{C} and AMH₂₅₅₋₅₆₀ In a reducing-conditions western blot were examined using antibodies to the N- and C-terminal portions of AMH (FIG. 3). The rhAMH derived from HEK cells was predominantly a 72 kDa species, with lesser amounts of other forms of AMH. The 72 kDa species is designated proAMH as it bound both the C- and N-terminal antibodies, and because pretreatment with the proprotein convertase furin diminished the intensity of the band. AMH_{C} at 12.5 kDa was detected exclusively by the C-terminal antibody, whereas two AMH_{N} bands at 62 and 64 kDa were detected exclusively by the N-terminal antibody. The Intensity of the AMH_{C} and AMH_{N} bands increased following furin treatment, providing further confirmation of their identities. A lower abundance 47 kDa band was also present. This band contained both N and C-terminal epitopes, with its size being consistent with the previously described AMH₂₅₅₋₅₆₀.

The level of AMH in blood is too low to be directly analyzed by western blots, necessitating prior concentration by immunoprecipitation. Applicants therefore first established that the non-specific bands associated with immunoprecipitation were distinct from the main AMH species. The proteins immunoprecipitated from human serum by a control goat IgG were different in size to proAMH, AMH_{N} and AMH_{C} (FIG. 4). Faint non-specific bands were detected at approximately 47 kDa. These bands would not obscure the presence of significant levels of AMH₂₅₅₋₅₆₀.

### Example 3

### AMH_{N,C} and ProAMH is Present in Human Blood

The blood of a boy was then examined by separate immunoprecipitation with anti-N- or anti-C-terminal antibodies to AMH, followed by separate detection with the anti-N- and anti-C-terminal antibodies. Significant levels of both proAMH and AMH_{N,C} were detected. The strongest band Immunoprecipitated by the N-terminal antibody had the characteristics of proAMH. That is, a reduced 72 kDa protein that reacts with both the anti-C and anti-N terminal antibodies (FIG. 5A,B). This band was not detected in the blot of a post-menopausal woman (FIG. 5A,B), who only had trace levels of AMH (Table 1).

The N-terminal antibody also immunoprecipitated bands with molecular weights appropriate for AMH_{N} (FIG. 5A) and AMH_{C} (FIG. 5B). Neither of these bands were present in the negative control (post-menopausal woman). The detection of AMH_{C} suggests it is co-immunoprecipitating as part of the AMH_{N,C} complex as the antibody to the N-terminal domain does not directly bind to AMH_{C} (FIG. 5A).

The C-terminal antibody immunoprecipitates rhAMH_{C} but does not precipitate protein from the rhAMH (FIG. 6), which is a mix of proAMH and AMH_{N,C} (FIG. 3). This antibody precipitated trace levels of AMH_{N} from the blood of the boy, with no detectable levels of either AMH_{C} or proAMH. This indicates that the boy's blood contained little or no free AMH_{C}, providing independent validation that the AMH_{C} precipitated by the anti-N antibody is due to the presence of AMH_{N,C} In blood. The trace AMH_{N} band suggests that the anti-C terminal antibody inefficiently precipitates AMH_{N,C}, with the greater sensitivity of the N- than the C-terminal detection permitting the visualisation of the precipitated AMH_{N}.

In subsequent experiments, the anti-N terminal was used for immunoprecipitation, with a combination of the anti-N and anti-C terminal antibodies used for the western blots to ensure that all precipitated species were detected.

### Example 4

### proAMH and AMH_{N,C} is Present in Adult Serum, from Both Sexes

Applicants then examined whether the presence of proAMH and AMH_{N,C} was dimorphic or dependent on the stage of the life cycle. AMH was immunoprecipitated from plasma samples from 2 additional pre-pubescent boys (B2, B3), 3 young men (M1, M2, M3) and 3 pre-menopausal women (W1, W2, W3), with a post-menopausal woman included as a control (FIG. 7). ProAMH and AMH_{N} were detected in the blood of the boys, men and women (FIG. 7) with the Intensity of the bands being broadly in line with the ELISA estimate of AMH levels (FIG. 7, Table 1). The postmenopausal woman had no detectable levels of AMH_{N}, and only trace levels of proAMH. The anti-N terminal antibody co-immunoprecipitated AMH_{C} from the serum of boys. The density of the AMH_{C} bands in the adult samples were low as expected (FIGS. 7 & 8A). The presence of AMH_{C} was therefore confirmed by densitometry (FIG. 8C), after which the visibility of the bands was made clearer by linear contrast enhancement (FIG. 8B). This indicates that AMH_{N,C} is present In adult blood, as well as the more easily detected proAMH.

TABLE 1
Study participant's age, sex and blood AMH concentration

| Group | Code | Age | Source | AMH pM (ng/ml)* | |
|---|---|---|---|---|---|
| Boys | B1 | 4 | plasma | 839 | (117) |
| | B2 | 5 | plasma | 320 | (44.8) |
| | B3 | 6 | plasma | 193 | (27.1) |
| | B4 | 8 | serum | 693 | (97.0) |
| | B5 | 7 | serum | 1260 | (176) |
| | B6 | 6 | serum | 1110 | (155) |
| | B7 | 5 | serum | 508 | (71.1) |
| Men | M1 | 24 | plasma | 69.9 | (9.78) |
| | M2 | 21 | plasma | 84.5 | (11.8) |
| | M3 | 22 | plasma | 76.9 | (10.8) |
| Women | W1 | 22 | plasma | 23.0 | (3.22) |
| | W2 | 22 | plasma | 27.9 | (3.90) |
| | W3 | 21 | plasma | 33.7 | (4.73) |
| Post-menopausal | PMW | 78 | plasma | 0.36 | (0.05) |
| woman | | | | | |

*AMH concentration was determined using conventional ELISA assay

### Example 5

### Glycosylation of Circulating AMH

The rhAMH had two AMH_{N} bands (FIG. 3), with some of the AMH_{N} bands precipitated from human blood having apparent minor variation in molecular weight. AMH has two putative N-linked glycosylation sites In the N-terminal region (Cate et al. (1986) Cell 45:685-698), which may account for this. Applicants therefore examined the glycosylation status of AMH in human blood, by investigating whether deglycosylation enzymes reduced the molecular weight of the proAMH and/or the AMH_{N} bands. A deglycosylation kit (Sigma Catalogue # E-DEGLY) was used to remove all N- and O-linked glycans This reduced the size of the proAMH and AMH_{N} bands, yielding several lower molecular weight species (FIG. 9).

The proAMH and AMH_{N} band in the blood of boys and adults run the same distance of gels (FIG. 7), suggesting that AMH is also glycosylated In adults. The lower abundance of AMH in adults makes this difficult to analyse In detail. However, enzyme deglycosylation of adult samples produced AMH_{N} and proAMH bands of lower molecular weight with higher intensity, in concordance with the changes observed with AMH from boys (FIG. 10).

### Example 6

### Conventional ELISA Assays Bind proAMH and AMH_{N,C}

To confirm the AMH species captured by existing commercial assays, Applicants employed the Beckman Coulter (BC ELISA; Catalogue # A79765).

Initially, unprocessed recombinant human AMH (rhAMH; which contains a mix of AMH species (FIG. 3)), recombinant human AMH_{C} (rhAMH_{C}) and recombinant human AMH_{N}(rhAMH_{N}) were added to separate ELISA wells. The wells were then washed using standard techniques according to the manufacturers instructions to remove any non-specifically bound AMH species. The captured proteins were then analyzed by western blot which included the recombinant proteins as a control (FIG. 11).

The BC ELISA plate bound proAMH, as evidenced by an appropriately sized band that was detected by both N-terminal and C-terminal antibodies to AMH (FIGS. 11A and 11B). This is an essential property of the capture antibody. In addition to proAMH, the BC ELISA matrix also captured AMH_{C} from the rhAMH_{C} preparation; AMH_{C}, AMH_{N}, and AMH₂₅₅₋₅₆₀ from rhAMH. The ELISA plate did not capture rhAMH_{N} from the rhAMH_{N} preparation.

The pattern of capture is consistent with the capture antibody being either an antibody to the C-terminal region (with the AMH_{N} being captured as part of AMH_{N,C}) or an antibody that recognises an epitope involving amino acids present on both AMH_{N} and AMH_{C}.

The capture of AMH_{N,C} Is not an essential feature of the proAMH ELISA, but is used here to demonstrate that the capture of AMH_{N,C} does not intefere with the measurement of proAMH.

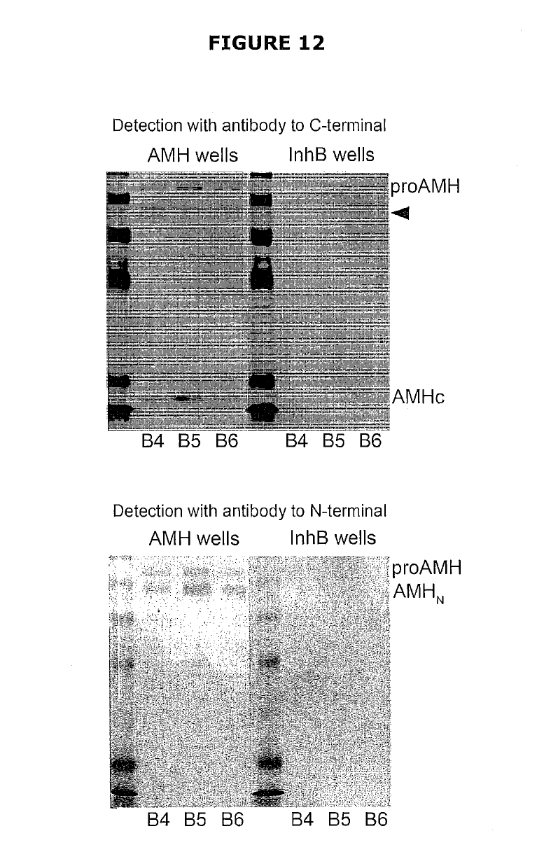

The same experiment was then conducted using serum samples from boys. Serum from three boys (B1-B3) was mixed with the commercial assay buffer and added to either AMH or Inhibin B (InhB) ELISA plates. The captured proteins were recovered and analyzed by western blots, using antibodies directed to the C- and N-terminals of AMH. The AMH ELISA plate captured proAMH, AMH_{C} and AMH_{N} from the blood of boys as evidenced by western blot analysis (FIG. 12). The western blot staining pattern observed for the serum sample obtained from boys was identical to that observed for the recombinant forms of AMH (FIG. 11), except that no AMH₂₅₅₋₅₆₀ was present. This is expected as blood does not contain detectable levels of AMH₂₅₅₋₅₆₀.

The antibody based specificity of the AMH (BC) ELISA plate for the various AMH species is evident from the control experiment conducted using InhB ELISA plate. As expected, the InhB ELISA plate failed to capture AMH, as evidenced by the lack of staining on the western blot (FIG. 12).

Since the BC ELISA works on C-terminal AMH capture, Applicants sought to confirm that similar results could be achieved using an assay which involves an N-terminal AMH capture assay. Accordingly, the ANSH ELISA was employed.

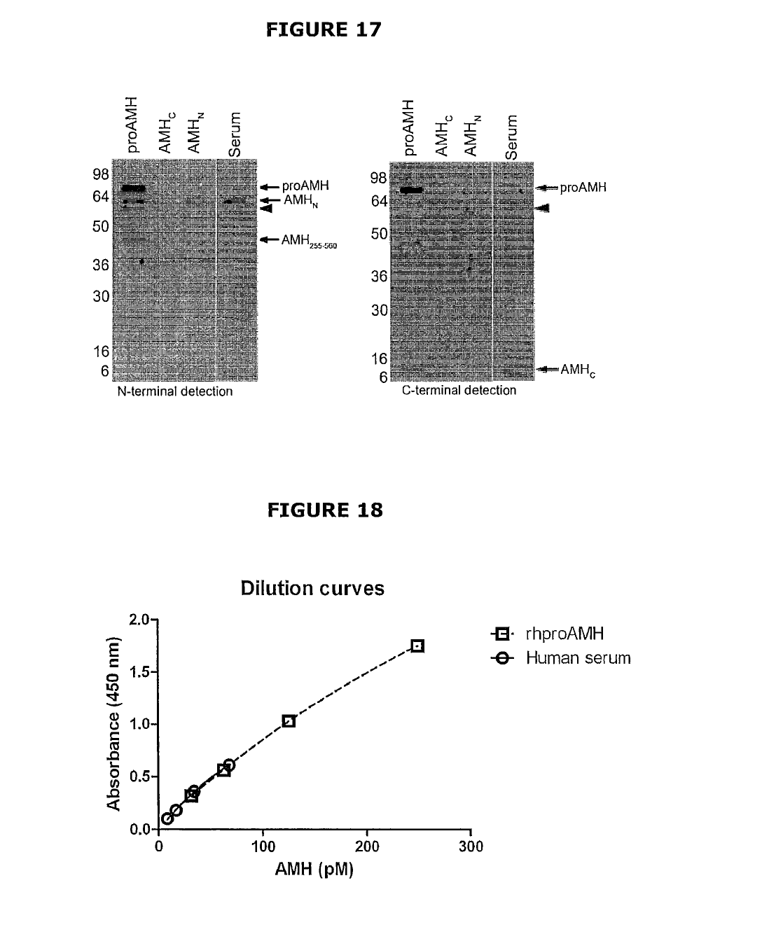

rhAMH_{C}, rhAMH_{N}, rhAMH or human serum were loaded onto wells from the Ansh Labs assay. The captured proteins were then recovered an analyzed by western blots using a detection antibody to either the N-terminal domain or C-terminal of AMH. The proAMH, AMH_{C}, AMH_{N} and AMH₂₅₅₋₅₆₀ bands are indicated on the western blot in FIG. 17, confirming that N-terminal capture will achieve the same outcome.

### Example 7

### Splitting of AMH_{N,C} Complex

The AMH_{N,C} complex may be split before addition of the sample to the assay matrix or after the AMH species have been captured.

### A. AMH_{N,C} Complex is Split Before Addition to the ELISA Matrix

Recombinant human AMH was treated with furin. Recombinant human AMH at 10 nM was incubated with 8 units of furin in 100 mM HEPES, pH 7.4, 1.8 mM Ca²⁺, 140 mM Na⁺, 4 mM K⁺, 0.05% v/v Tween-20. The reaction was allowed to proceed at 37° C. for 24 hours to produce the cleaved form of AMH (rhAMH_{N,C}), with only trace levels of proAMH present.

Varying concentrations of sodium deoxycholate at 0.05%, 0.1% or 0.2% w/v were added to rhAMH or rhAMH_{N,C} samples, and the samples incubated at 37° C. Following deoxycholate treatment, the samples were then added to an ELISA plate (Beckman Coulter; Catalogue # A79765).

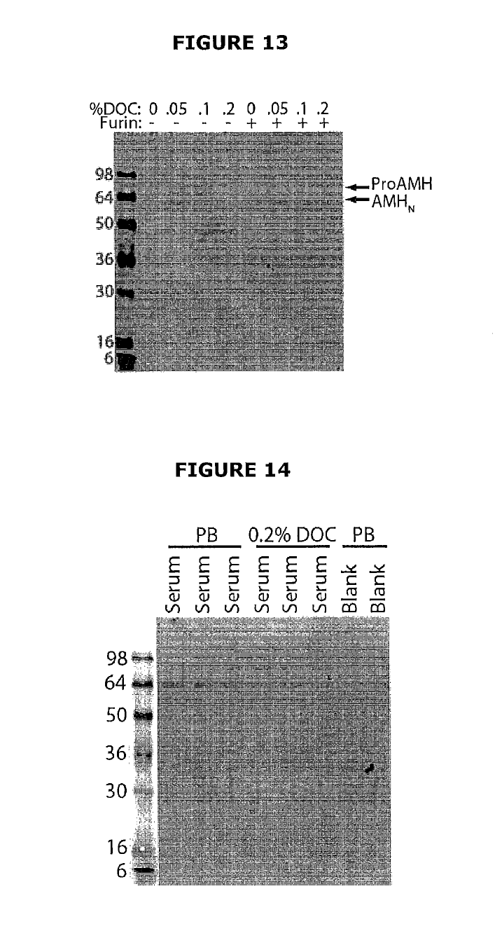

0.1% deoxycholate prevented the capture of the AMH_{N} present in the rhAMH_{N,C}, but did not prevent the capture of proAMH (FIG. 13). This is consistent with the ELISA plate comprising a capture antibody specific for the C-terminal domain of AMH.

Increasing the concentration of deoxycholate (e.g. >0.1% w/v) disrupted the binding of all species of AMH, as evidenced by the lack staining in the western blot (FIG. 13; 0.2% DOC). This highlights the Importance of optimising the amount or concentration of the splitting reagent used In the assay. For example, if the concentration of the splitting reagent (in this case deoxycholate) is too strong, it will cause disruption to the binding interaction between proAMH and the ELISA plate. Conversely, if the concentration of the splitting reagent is too weak, this will cause incomplete splitting of the AMH_{N,C} complex. Either scenario will affect the sensitivity of the assay and quantitative measure of proAMH.

B. AMH_{N,C} Complex is Split after Addition to the ELISA Matrix

In this experiment, an aliquot of serum obtained from an adult male was added to an ELISA plate (Beckman Coulter; Catalogue # A79765) according to the manufacturers instructions (sample added; followed by 100 μL of BC assay buffer and the wells incubated for 1 hour at room temperature, to enable capture of the AMH species to occur).

Serum was added to a total of six wells, and three of the wells were then washed twice with 0.2% deoxycholate, prewarmed to 37° C. for 15 minutes at 37° C. per wash.

Importantly, the deoxycholate wash released the captured AMH_{N}, without loss of the captured proAMH, as evidenced by western blot (FIG. 14). Note: a higher concentration of deoxycholate (0.2%) was used to split the AMH_{N,C} complex when bound to the ELISA plate, compared to when the AMH_{N,C} complex is split prior to its capture (example above). The required concentration of the spitting reagent will be affected by temperature, time and context (i.e. the sample to which it is added). The optimal concentrations of splitting reagent for splitting prior to addition to the ELISA plate, and after the capture step need to be separately determined.

### Example 8

### Demonstration of a Working proAMH ELISA

To demonstrate a working proAMH specific ELISA, Applicants modified existing conventional AMH ELISAs to include a deoxycholate wash step following protein capture, namely, the Beckman Coulter (BC ELISA; Catalogue #A79765) and the ANSH Labs AMH ELISA (ANSH ELISA; Catalogue #A79765). As shown below, this modification changed the ELISA from an assay specific for the sum of proAMH+AMH_{N,C}, to an assay which is specific for proAMH.

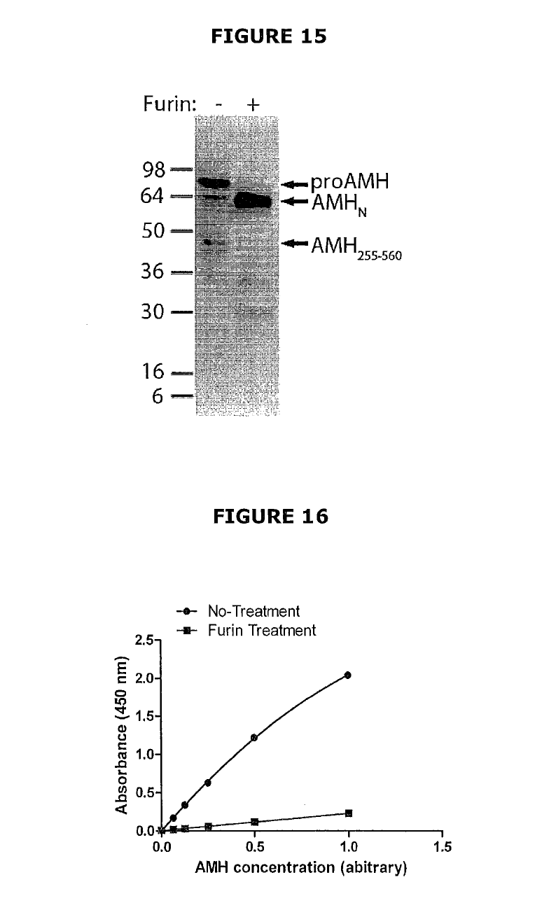

An aliquot of rhAMH was cleaved with furin to generate rhAMH_{N,C} (FIG. 15). The amount of AMH measured using the unmodified BC ELISA did not significantly change when rhAMH (proAMH; 18.7 pM) was converted rhAMH_{N,C} (19.0 pM). Refer to Table 2. This provides further direct evidence that conventional ELISAs measure both proAMH and AMH_{N,C}.

TABLE 2
The BC genII ELISA detects proAMH and AMH_{N, C}

| AMH species | BC ELISA (pM) |
|---|---|
| rhAMH (proAMH) | 18.7 |
| rhAMH_{N, C}* | 19.0 |
| AMH_{C} | 0.0 |
| AMH_{N} | 0.0 |

*rhAMH_{N, C} is furin-treated rhAMH (FIG. 15)

The BC ELISA did not detect rhAMH_{C} and rhAMH_{N}, which In combination with the above observation shows that the detection and capture antibodies recognise epitopes on different termini of AMH (i.e. N-terminal and C-terminal domains).

When rhAMH (proAMH) was assayed using the deoxycholate-modified ELISA then a dose-dependent output was obtained (FIG. 16). Equivalent concentrations of rhAMH_{N,C} (furin-treated rhAMH) produced only a small signal, which is most likely due to the trace levels of proAMH in the preparation.

### Example 9

### proAMH ELISA Detects proAMH in Human Samples

Serial dilution of serum AMH shows a parallel relationship to serial dilution of the rhproAMH standard in the proAMH ELISA. Serum from a human male underwent 1:1 serial dilution in an Identical manner to the standard curve. The concentration of the undiluted human serum was interpolated from the standard curve and the concentrations of AMH in the diluted serum were calculated using the dilution factor. FIG. 18 shows that the ELISA detects AMH from human samples and there is no difference in detection compared to the rhproAMH used for the standard.

### Example 10

### AMH_{N,C} Specific ELISA

To demonstrate a working AMH_{N,C} specific ELISA, Applicants will modify existing ELISA formats, for example the Beckman Coulter assay (Catalogue #A79765), to include a deoxycholate wash step either before or after protein capture. This modification will change the ELISA from an assay specific for the sum of proAMH+AMH_{N,C}, to an assay which is specific for AMH_{N,C}.

In one embodiment, the assay will employ a detection antibody which specifically binds to AMH_{C} but not to proAMH. This is demonstrated in FIG. 6 which shows that, under immunoprecipitation conditions, the detection antibody (Goat anti-rat AMH/MIS antibody (R&D systems, Catalogue# AF1446)) binds to rhAMH_{C} (lane 2), but not to rhAMH (proAMH; lanes 3 and 4). Since the ELISA will be performed under similar conditions (e.g. non-reducing/non-denaturing/soluble conditions very similar to the immunoprecipitation conditions used In FIG. 6), it is expected that the detection antibody will bind to and detect only AMH_{C}. The amount of bound AMH_{C} will be directly proportional to the amount of AMH_{N,C} (i.e. that which existed prior to the splitting step).

### Example 11

### Demonstration of a Working AMH_{N,C} ELISA

Applicants sought to demonstrate a working AMH_{N,C} ELISA by modifying an existing assay format, namely R&D Systems duoset ELISA (Cat #DY1737), specific for AMH_{C}.

Under normal conditions the AMH duoset ELISA detects AMH_{C} with high efficiency, although it does not detect proAMH and the signal is greatly diminished for AMH_{N,C} (FIG. 19).

The assay results are evident from FIG. 21, which demonstrates that the ELISA detects AMH_{C} or AMH_{N,C}, but not proAMH. Varying concentrations (nM) of non-treated rhMAH (proAMH with small quantities of AMH_{N,C}) and furin treated rhMAH (AMH_{N,C} with small quantities of proAMH) were assayed following a deoxycholate pre-treatment step (Example 1).

Only AMH_{C}, i.e. not proAMH or AMH_{N}, can be recovered from the multi-well plate. This suggests that proAMH and AMH_{N,C} are sterically hindered from binding to the capture antibody. The weak detection of proAMH is explained by the sample containing small quantities of AMH_{N,C}. Similarly, the weak detection of AMH_{N,C} must occur due to some degree of dissociation and reassociation between AMH_{N} and AMH_{C} In the AMH_{N,C} complex. By pre-treating samples with 0.2% deoxycholate, the AMH_{N,C} complex dissociates allowing the AMH_{C} fragment to bind to the ELISA capture antibody. Free-AMH_{C} Is not known to exist in human serum and therefore any AMH detected by this ELISA after deoxycholate pre-treatment of serum samples will be AMH_{N,C} and not proAMH.

### Example 12

### Relative Abundance of proAMH and AMH_{N,C} in the Blood of Healthy Individuals

Applicants sought to describe the relative abundance of proAMH and AMH_{N,C} in the blood of healthy individuals recruited from the community. The study incorporates a comparison between the sexes and between the major stages of the life cycle for each sex.

Children were recruited via flyers distributed to local schools and advertisements In local newspapers and adult participants were recruited from the community via flyers and newspaper advertisements in Dunedin, New Zealand.

The male dataset contained: 131 boys aged from 4 to 11 years old; 100 men aged between 24 and 65 years; 101 elderly men, who were older than 65 years (Table 3). The female dataset contained: 20 girls aged from 4 to 11 years old; 34 women aged between 19 and 31 years old and 25 women aged between 31 and 46 years old (Table 3). The stratification at the age of 31 years delineates the ages of peak and diminishing fertility (http://www.ncbl.nlm.nlh.gov/pubmed/19589949). Elderly women were not examined as they lack detectable levels of total AMH (Chong et al. (2012) Int J Gynecol Cancer 22:1497-1499).

Total AMH was measured using Beckman Coulter's AMH Gen II ELISA and standards, according to the manufacturer's instructions, in accordance with field safety notice FSN-20434-3, June 2013. The serum of boys was diluted 1:9 In sample diluent before assay.

ProAMH was quantified with the proAMH assay as described in Examples 8 and 9, using the AMH Gen II ELISA plate, detection reagents and ELISA standards.

TABLE 3
Population age characteristics

| | n | Range | Mean | SD | Skewness | Kurtosis |
|---|---|---|---|---|---|---|
| Females | | | | | | |
| Girls (4-11) | 20 | 4.35-10.1 | 7.6 | 1.4 | −0.62 | 0.99 |
| Women (19-31) | 34 | 19.0-30.0 | 23.4 | 3.2 | 0.75 | 0.79 |
| Women (31-46) | 13 | 31.0-46.4 | 39.2 | 4.5 | 0.41 | 0.90 |
| Males | | | | | | |
| Boys (4-11) | 131 | 4.5-10.5 | 6.7 | 1.1 | 0.67 | 1.16 |
| Men (24-65) | 101 | 24.5-64.1 | 43.7 | 9.4 | 0.26 | −0.67 |
| Men (>65) | 91 | 66.5-89.7 | 77.6 | 5.2 | 0.21 | −0.29 |

The proportion of total AMH that was proAMH was calculated as defined above. When a ratio of two numbers close to zero is taken, the resulting number is very sensitive to any imprecision in the original numbers, particularly the denominator. ELISA values near the limit of detection have elevated error. For this reason, the % proAMH values were not calculated when an individual's total AMH levels were less than 10 pM. The following number of participants were excluded by this criteria: 0 boys; 0 younger men; 6 elderly men; 3 girls; 3 younger women and 13 women from the 31-46 age group.

The results for each sex and age group are summarised below, and captured in Tables 4 and 5, as well as FIGS. 23 and 24.

The key points to note are:
1. proAMH levels can be measured In human serum;
2. the levels of proAMH, total AMH and % proAMH vary between Individuals, and between distinct population groups; and
3. The proAMH ELISA generates data that is distinct from the existing AMH ELISAs

### A. Boys

The level of proAMH in boys was invariably high, but with an Inter-person variation that spanned an order of magnitude. The minimum level of proAMH in the 131 boys was 74.9 pM, a value that was over double the maximum value in all other population groups (Table 4).

The % proAMH in boys exhibited biological variation, with a mean of 33% and a standard deviation of 4.4% (Table 5). The ranges of % proAMH in the boys and the men samples did not overlap, indicating that the relative levels of proAMH and AMH_{N,C} change markedly during the pubescent transition.

### B. Men

The absolute levels of proAMH and the % proAMH in the samples from men were significantly lower than those for boys (Tables 4, 5). The absolute level of proAMH was variable between men, with a range of 2.1 to 33.5 pM. This range has extensive overlap with that the range for women. The % proAMH in men tended, however, to be lower than in women.

### C. Elderly Men

Ten elderly men with a mean age of 80.2±5.4 SD had proAMH levels that were below the limit of detection, which was set at 1.14 pM. The mean level of proAMH in the elderly men with detectable levels of proAMH was lower than for the younger men (Table 4), despite the ten lowest values being excluded. This indicates that the level of proAMH in the blood of men either tends to decrease as they age or that there is a secular trend for more recent generations to have higher levels of proAMH. The mean % proAMH in the elderly men, in contrast, was not different to that of the younger men. The distribution of the % proAMH in the young and elderly men was also very similar, with the exception of a single older man who had a % proAMH level that approached the boy range (Table 5). Note that % proAMH could not be calculated for 16% of the elderly men with the lowest levels of proAMH (n=6) and total AMH (n=10).

### D. Girls

Seventeen of the 20 prepubescent girls had undetectable levels of proAMH (Table 4). The level of proAMH in the other 17 girls were similar to levels in young women. In contrast, the % proAMH levels in the girls was highly variable: the lower end of the range extensively overlapped with that of women, with the upper range end overlapping with the boy range (Table 5).

### E. Women

The levels of proAMH in young women were little different to those of girls. All women had detectable levels of proAMH, but the women in the 31-46-year-old group had significantly less proAMH, on average. This contrasts with the women's level of total AMH which were higher in young women than in girls (Table 4).

The % proAMH range in the young women was lower and narrower than that of the girls. Older women had a higher mean % proAMH value than younger women, with the range of % proAMH levels trending towards that of girls (Table 5).

These data indicate that significant changes occur in the relative levels of proAMH and AMH_{N,C} at the beginning and end of reproductive life. The rise in total AMH during puberty involves both AMH species, but with a preferential increase in AMH_{N,C}, whereas the entry to menopause appears to involve the mirror image of this, with total AMH, proAMH and AMH_{N,C} all declining, but with the average loss of AMH_{N,C} being greater than the loss of proAMH.

TABLE 4
Levels of proAMH and total AMH in the study populations

| Group | | n | Minimum | Maximum | Mean | Std. Dev | Skewness | Kurtosis |
|---|---|---|---|---|---|---|---|---|
| Boys | proAMH | 131 | 74.9 | 674.4 | 253.1 | 100.2 | 0.91 ± 0.21 | 1.9 ± 0.4 |
| | Total AMH | 131 | 192.5 | 1708.1 | 779.1 | 306.0 | 0.43 ± 0.21 | −0.1 ± 0.4 |
| Men | proAMH | 100 | 2.1 | 33.5 | 7.3 | 4.6 | 2.64 ± 0.24 | 10.9 ± 0.5 |
| | Total AMH | 100 | 10.2 | 147.2 | 41.3 | 24.7 | 1.97 ± 0.24 | 5.3 ± 0.5 |
| Elderly Men | proAMH | 91 (10) | n.d. | 15.8 | 4.9 | 3.2 | 1.80 ± 0.25 | 3.5 ± 0.5 |
| | Total AMH | 91 (10) | n.d. | 103.5 | 30.0 | 20.9 | 1.73 ± 0.25 | 3.1 ± 0.5 |
| Girls | proAMH | 21 | 5.6 | 16.0 | 8.8 | 2.7 | 1.20 ± 0.50 | 1.1 ± 0.1 |
| | Total AMH | 21 | 5.8 | 55.8 | 22.2 | 11.9 | 1.29 ± 0.50 | 2.0 ± 1.0 |
| Women 19-31 y | proAMH | 34 | 2.5 | 21.5 | 9.0 | 4.9 | 0.86 ± 0.40 | 0.1 ± 0.8 |
| | Total AMH | 34 | 8.1 | 93.7 | 33.9 | 21.5 | 1.15 ± 0.40 | 0.8 ± 0.8 |
| Women 31-46 y | proAMH | 25 | 2.6 | 13.3 | 5.1 | 2.6 | 1.58 ± 0.46 | 2.6 ± 0.9 |
| | Total AMH | 23 | 1.0 | 53.4 | 14.5 | 12.2 | 1.62 ± 0.48 | 3.4 ± 0.9 |

TABLE 5
% proAMH in the study populations

| | n | Range | Mean | SD | Skewness | Kurtosis |
|---|---|---|---|---|---|---|
| Females | | | | | | |
| Girls (4-11) | 17 | 26.8-56.1 | 40.8^{a} | 8.1 | 0.61 | −0.18 |
| Women (19-31) | 31 | 19.3-40.1 | 27.6^{b} | 4.8 | 0.86 | 0.81* |
| Women (31-46) | 13 | 22.0-47.8 | 33.6^{c} | 7.6 | 0.23 | −0.81 |
| Males | | | | | | |
| Boys (4-11) | 131 | 24.9-49.2 | 33.0^{x} | 4.4 | 0.90 | 1.23* |
| Men (24-65) | 100 | 11.1-24.8 | 17.8^{y} | 2.8 | 0.29 | −0.27 |
| Men (>65) | 91 | 11.1-36.2 | 17.1^{y} | 3.6 | 2.50 | 10.7* |

*Non-normal distribution as determined by Kolrnogorov-Smirnov test
^{a,b,c}Shared letters indicate lack of significant difference between female groups (1-way ANOVA)
^{x,y}Shared letters indicate lack of significant difference between male groups (1-way ANOVA)

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference In its entirety individually or set forth herein in its entirety. Applicants reserve the right to physically incorporate into this specification any and all materials and Information from any such patents, publications, scientific articles, web sites, electronically available information, and other referenced materials or documents.

Although the Invention has been described by way of example, it should be appreciated that variations and modifications may be made without departing from the scope of the Invention as defined in the claims. Furthermore, where known equivalents exist to specific features, such equivalents are incorporated as if specifically referred In this specification. The specific assays and methods described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the Invention disclosed herein without departing from the scope and spirit of the Invention. The invention illustratively described herein suitably may be practiced in the absence of any element or elements, or limitation or limitations, which is not specifically disclosed as essential. Thus, for example, in each instance described or used herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The assays and methods illustratively described herein suitably may be practiced in differing orders of steps, and that they are not necessarily restricted to the orders of steps indicated herein or in the claims. Further, as used or described herein and In the appended claims, the singular forms “a,” “an,” and “the” Include plural reference unless the context clearly dictates otherwise. Under no circumstances may the patent be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the Invention as claimed. Thus, it will be understood that although the present invention has been specifically disclosed by preferred embodiments and optional features, modification and variation of the concepts disclosed herein may be resorted to by those skilled in the art, and that such modifications and variations are considered to be within the scope of this invention as described herein, and as defined by the appended claims.

The Invention has been described broadly and generically herein. Each of the narrower species and subgeneric groupings falling within the generic disclosure also form part of the Invention. This includes the generic description of the invention with a proviso or negative limitation removing any subject matter from the genus, regardless of whether or not the excised material is specifically recited herein.

Other embodiments are within the following claims. In addition, where features or aspects of the Invention are described in terms of Markush groups, those skilled in the art will recognize that the Invention is also thereby described in terms of any individual member or subgroup of members of the Markush group.

1. An assay for proAMH in a biological sample comprising proAMH and AMH_{N,C} obtained from a subject, the assay comprising measuring the level of proAMH in the sample using a first binding agent, a second binding agent and a splitting reagent, wherein:
(i) the splitting reagent splits the AMH_{N,C} complex into AMH_{N} and AMH_{C}; and
(ii) when the first binding agent binds to the N-terminal domain of proAMH and AMH_{N,C}, the second binding agent is specific for and binds to the C-terminal domain of proAMH; or
(iii) when the first binding agent binds to the C-terminal domain of proAMH and AMH_{N,C}, the second binding agent is specific for and binds to the N-terminal domain of proAMH.

2. An assay according to claim 1, further comprising:
(i) contacting the biological sample with the first binding agent such that it binds to proAMH and AMH_{N,C};
(ii) contacting the bound proAMH and AMH_{N,C} with the splitting reagent in an amount sufficient to split the AMH_{N,C} complex into AMH_{N} and AMH_{C}; and
(iii) contacting the bound proAMH and AMH_{N} or AMH_{C} with the second binding agent specific for proAMH and measuring the level of bound proAMH.

3. An assay according to claim 1, further comprising:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent specific for proAMH and measuring the level of bound proAMH.

4. An assay according to any one of claims 1 to 3, wherein the first binding agent binds to the C-terminal domain of AMH and the second binding agent binds to the N-terminal domain of AMH.

5. An assay according to any one of claims 1 to 3, wherein the first binding agent binds to the N-terminal domain of AMH and the second binding agent binds to the C-terminal domain of AMH.

6. An assay for AMH_{N,C} In a biological sample comprising proAMH and AMH_{N,C} obtained from a subject, the assay comprising measuring the level of AMH_{N,C} in the sample using a first binding agent, a second binding agent and a splitting reagent, wherein the first binding agent binds to proAMH and AMH_{N,C}, the second binding agent binds to AMH_{N} or AMH_{C} but does not bind to proAMH or AMH_{N,C} and wherein the splitting reagent splits the AMH_{N,C} complex into AMH_{N} and AMH_{C}.

7. An assay according to claim 6, further comprising:
(i) contacting the biological sample with the first binding agent such that it binds to proAMH and AMH_{N,C};
(ii) contacting the bound proAMH and AMH_{N,C} with the splitting reagent In an amount sufficient to split the AMH_{N,C} complex into AMH_{N} and AMH_{C}; and
(iii) contacting the bound proAMH and AMH_{N} or AMH_{C} with the second binding agent and measuring the level of bound AMH_{N} or AMH_{C},
wherein the level of bound AMH_{N} or AMH_{C} corresponds to the amount of AMH_{N,C} In the sample.

8. An assay according to claim 6, further comprising:
(i) contacting the biological sample with the splitting reagent in amount sufficient to split the AMH_{N,C} complex into AMH_{N} and AMH_{C};
(ii) contacting the biological sample with the first binding agent such that the first binding agent binds to proAMH and AMH_{N} or AMH_{C}; and
(iii) contacting the bound proAMH and AMH_{N} or AMH_{C} with the second binding agent and measuring the level of bound AMH_{N} or AMH_{C},
wherein the level of bound AMH_{N} or AMH_{C} corresponds to the amount of AMH_{N,C} In the sample.

9. An assay according to any one of claims 1 to 8 wherein the first binding agent is immobilised on a solid support.

10. An assay according to any one of claims 1 to 9 wherein the second binding agent comprises a detectable label.

11. An assay according to any one of claims 1 to 10, wherein the first binding agent is an antibody or an antigen binding fragment thereof.

12. An assay according to any one of claims 1 to 11, wherein the second binding agent is an antibody or an antigen binding fragment thereof.

13. An assay according to claim 11 or claim 12, wherein the antibody is selected from the group consisting of a polyclonal, monoclonal, bispecific, chimeric and humanized antibody or an antigen binding fragment thereof.

14. An assay according to claim 13, wherein the antibody is a monoclonal antibody or antigen binding fragment thereof.

15. An assay according to any one of claims 6 to 8, wherein the first binding agent or second binding agent is a labelled form of AMH_{N} or AMH_{C}.

16. An assay according to any one of claims 1 to 15, wherein the splitting reagent is selected from the group consisting of a salt, a detergent, and a chemical reagent sufficient to alter pH.

17. An assay according to claim 16, wherein the chemical reagent sufficient to alter pH comprises an acid or a base.

18. An assay according to claim 16, wherein the splitting reagent comprises deoxycholate.

19. An assay according to any one of claims 1 to 18, wherein the assay is selected from the group consisting of an enzyme linked immunosorbent assay (ELISA), a radioimmunoassay (RIA), a fluoroimmunoassay, an immunofluorometric assay, and an immunoradiometric assay.

20. An assay according to any one of claims 1 to 19, wherein the biological sample is selected from the group consisting of a whole blood sample, serum, plasma, an ovarian follicular fluid sample and a semen sample.

21. An assay according to any one of claims 1 to 20 wherein the subject is a human, primate, farm animal including cattle, sheep, goat, pig, deer, alpaca, llama, buffalo, companion and/or pure bred animal including cat, dog and horse.

22. A dual purpose assay for measuring the levels of different species of AMH in a biological sample, the dual purpose assay comprising any combination of the assays according to any one of claims 1 to 21.

23. The dual purpose assay according to claim 22 which measures the levels of proAMH and AMH_{N,C}.

24. A kit for measuring proAMH in a biological sample obtained from a subject comprising proAMH and AMH_{N,C}, the kit comprising a first binding agent, a second binding agent, a splitting reagent and Instructions for how to measure the level of proAMH in the sample.

25. A kit according to claim 24, wherein:
(i) the splitting reagent is capable of splitting the AMH_{N,C} complex into AMH_{N} and AMH_{C}; and
(ii) when the first binding agent binds to the N-terminal domain of proAMH and AMH_{N,C}, the second binding agent is specific for and binds to the C-terminal domain of proAMH; or
(iii) when the first binding agent binds to the C-terminal domain of proAMH and AMH_{N,C}, the second binding agent is specific for and binds to the N-terminal domain of proAMH.

26. A kit for measuring AMH_{N,C} In a biological sample comprising proAMH and AMH_{N,C} obtained from a subject, the kit comprising a first binding agent, a second binding agent, a splitting reagent, and Instructions for how to measure the level of AMH_{N,C} in the sample.

27. A kit according to claim 26, wherein:
(i) the splitting reagent splits the AMH_{N,C} complex into AMH_{N} and AMH_{C}; and
(ii) when the first binding agent binds to proAMH and AMH_{N,C}, the second binding agent is specific for and binds to AMH_{N} or AMH_{C}.

28. A kit according to any one of claims 24 to 27, wherein the first binding agent is immobilised on a solid support.

29. A kit according to any one of claim 24 to claim 28, wherein the second binding agent comprises a detectable label.

30. A kit according to any one of claims 24 to 29, wherein the first and second binding agent is an antibody or antigen binding fragment thereof.

31. A kit according to claim 30, wherein the antibody is selected from the group consisting of a polyclonal, monoclonal, bispecific, chimeric and humanized antibody or an antigen binding fragment thereof.

32. A kit according to claim 26 or claim 27, wherein the first binding agent or the second binding agent is a labelled form of AMH_{N} or AMH_{C}.

33. A kit according to any one of claims 24 to 32 wherein the splitting reagent comprises deoxycholate.

34. A method for measuring the level of proAMH in a subject, the method comprising:
(i) obtaining a biological sample from the subject; and
(ii) measuring the level of proAMH in the sample using a first binding agent, a second binding agent and a splitting reagent.

35. A method according to claim 34, wherein the splitting reagent splits the AMH_{N,C} complex into AMH_{N} and AMH_{C}, and wherein when the first binding agent binds to the N-terminal domain of proAMH and AMH_{N,C}, the second binding agent is specific for and binds to the C-terminal domain of proAMH, or wherein when the first binding agent binds to the C-terminal domain of proAMH and AMH_{N,C}, the second binding agent is specific for and binds to the N-terminal domain of proAMH.

36. A method for measuring the level of AMH_{N,C} in a subject, the method comprising:
(i) obtaining a biological sample from the subject; and
(ii) measuring the level of AMH_{N,C} in the sample using a first binding agent, a second binding agent and a splitting reagent.

37. A method according to claim 36 wherein the first binding agent binds to proAMH and AMH_{N,C}, the second binding agent binds to AMH_{N} or AMH_{C} but does not bind to proAMH or AMH_{N,C}, and wherein the splitting reagent is sufficient to split the AMH_{N,C} complex into AMH_{N} and AMH_{C}.
